(19) United States
(12) Patent Application Publication
(10) Pub. No.: US 2023/0382955 A1
(43) Pub. Date: Nov. 30, 2023

(54) Method, Compositions and Applications of Mechanosensitive Channels

(71) Applicant: Nanoscope Technologies LLC, Bedford, TX (US)

(72) Inventors: Samarendra Kumar Mohanty, Arlington, TX (US); Subrata Batabyal, Arlington, TX (US); Darryl Narcisse, Crowley, TX (US); Adnan Dibas, Arlington, TX (US); Chinenye Abiodun Idigo, Mansfield, TX (US)

(21) Appl. No.: 18/151,752

(22) Filed: Jan. 9, 2023

Related U.S. Application Data
(63) Continuation-in-part of application No. 17/419,878, filed on Jun. 30, 2021, now Pat. No. 11,548,920.
(60) Provisional application No. 62/786,955, filed on Dec. 31, 2018.

(51) Int. Cl. C07K 14/195; A61P 43/00

(52) U.S. Cl. CPC C07K 14/195; A61P 43/00

(57) ABSTRACT

The present invention generally relates to the novel method, compositions and applications of mechanosensitive channels in therapeutics for visual, neurological and other disorders. Specifically, the invention relates to exploitation of mechanosensitive channels' intrinsic property as a transgenic pressure modulator and alternative outflow actuator in the treatment of different diseases such as glaucoma, blood pressure and other diseases involving of cells prone to mechanical or osmotic stress. Furthermore, the invention relates to exploitation of mechanosensitive channels for stimulation of cells enabling therapies for different diseases including pain, as well as molecular delivery to cells triggered by internal and/or external mechanical stimuli.

### CROSS-REFERENCE

This application is a continuation-in-part of U.S. patent application publication no. US2022/0048956A1 filed on Dec. 30, 2019, which in turn claims the benefit of U.S. provisional application No. 62/786,955 filed Dec. 31, 2018, all of which are hereby incorporated herein by reference their entirety.

Some references, which may include publications, patents, and patent applications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference were individually incorporated by reference.

### DESCRIPTION OF THE TEXT FILE SUBMITTED ELECTRONICALLY

The contents of the text field submitted electronically herewith are incorporated herein by reference in their entirety: A computer readable format copy of the Sequence Listing (file name: SIP010-0005US-I1—sequence listing ST.26.xml, date recorded: Jan. 9, 2023, file size 13 kilobytes) which is a ST.26.xml conversion of the Sequence Listing filed on Jun. 10, 2022 (file name: Chinyenye_SL_revised_June 2022.txt, date recorded: Jun. 10, 2022, file size 10 kilobytes), which in turn replaced Sequence Listing (CHINENYE_SL_2019.txt, date recorded: Dec. 4, 2019, file size 9 kilobytes), which are all incorporated herein by reference in their entirety.

### STATEMENT REGARDING FEDERALLY SPONSORED RESEARCH OR DEVELOPMENT

This invention was made with funding by Nanoscope Technologies, LLC. The Government has no rights in the invention.

### FIELD OF INVENTION

The present invention generally relates to the novel method, compositions, and applications of mechanosensitive channels in therapies of different disorders of eye and other organs. Specifically, the invention relates to exploitation of mechanosensitive channels of large conductance (MscL)'s intrinsic property as a transgenic pressure modulator and alternative outflow actuator in the treatment of glaucoma, dry-eye and other diseases involving of cells prone to mechanical or osmotic stress. Furthermore, the invention relates to exploitation of mechanosensitive channels for molecular delivery triggered by internal as well as external mechanical stimuli.

### BACKGROUND OF INVENTION

Cells of different organs in the body undergoes range of mechanical and osmotic pressures which changes in various diseases including neurological, cardiovascular, ophthalmological, and renal diseases. While in bacteria, the mechanosensitive channel of large conductance (MscL) functions as an osmoregulator and protects cells from lysis upon hypo-osmotic shock, such channels are not present in mammalian cells.

As an example of a targeted pressure-related disorder, Vision loss due to glaucoma is the second leading cause of blindness in America¹. The livelihood, independence, and quality of life of millions of glaucoma patients and their families are being disrupted by this pervasive disease.

Glaucoma is defined as a group of eye conditions, which cause damage to the optic nerve usually due to elevated intraocular pressure (IOP). The most common form of glaucoma is primary open angle glaucoma (POAG) and it is characterized by poor drainage of aqueous humor through the conventional outflow pathway. In the conventional outflow pathway, aqueous humor drains through the trabecular meshwork (TM), into Schlemm's canal and on into the episcleral vein. Several studies have characterized distinct changes in TM cellular processes and proteinaceous structures in glaucomatous eyes, which purportedly account for increased TM stiffness and aqueous humor outflow resistance².

The eye must maintain a healthy internal pressure to preserve its form and function. A healthy intraocular pressure (IOP) is maintained by continuous upkeep of vitreous and aqueous humor processing. Aqueous humor is responsible for the structure, nutrition, and hygiene of the tissues in the anterior chamber and proper regulation of its inflow and outflow balance is critical. Aqueous humor is produced by the ciliary body, flows through the pupil, and drains out through either the conventional outflow pathway or the uveoscleral pathway. In the conventional outflow pathway aqueous humor flows through the trabecular meshwork (TM), into Schlemm's canal, and out into episcleral blood vessels through ducts that branch of the canal. Glaucoma is defined as a group of eye conditions which cause damage to the optic nerve, usually due to elevated IOP. There are four main avenues by which IOP can be decreased; i) reduce aqueous humor production, ii) increase outflow through the uveoscleral pathway, iii) improve drainage through the conventional pathway, or iv) create an alternate outflow pathway.

The currently available pharmacological and surgical treatments for glaucoma have significant limitations and side effects. These undesirable side effects include systemic reactions to medications, patient non-compliance, eye infections, surgical device failure, and damage to other structures of the eye. To forestall disease progression, most glaucoma cases will need multiple medical interventions over time. For example, even after surgery some glaucoma patients will still need to take daily eyedrops for an indefinite period. The development of a safe effective long-lasting single-dose therapeutic for the ubiquitous treatment of glaucoma.

The other treatment option for uncontrollable glaucoma is surgery. The most common glaucoma surgery is a trabeculectomy, where an alternative outflow pathway is created by removing a section of the sclera, Schlemm's canal and TM and fashioning a filtration bleb out of a conjunctival tissue flap. Other surgical methods include, canaloplasty, laser trabeculoplasty, laser peripheral iridotomy, deep non-perforating sclerectomy, and drainage device implantation. As with all surgeries, there are risks of infection, systemic side effects and human/device failure. Post-surgical scarring at the procedure site is common and scar tissue can block or shift the newly created drainage pathway. With the added considerations of the expense, patient recovery time and inconsistent outcomes of surgery, it becomes clear that new and innovative treatments for glaucoma are needed.

### SUMMARY OF THE INVENTION

To meet the challenges, the present invention provides novel methods, compositions, and applications of heterologously expressed mechanosensitive channels as tension activated pressure release valves in mammalian cells including trabeculocytes and epithelial cells.

In bacteria, the mechanosensitive channel of large conductance (MscL) functions as an osmoregulator and protects cells from lysis upon hypo-osmotic shock. MscL directly senses tension in the membrane lipid bilayer of the swelling cell and in response transiently opens its large non-specific pore to release cytoplasmic fluid, thereby relieving the building turgor pressure.

In an embodiment of the invention, the present invention describes a method to use MscL as a virally delivered transgenic pressure modulator in the impaired TM cells of glaucomatous eyes.

In an embodiment of the invention, the present invention describes a synthetic polypeptide sequence of MscL1 protein and its generated site-directed mutants comprising: An MscL1 protein (SEQ ID NO: 1) that, when expressed on mammalian cell membrane, senses pressure changes and modulate the intra-cellular pressure, or molecular transport not limited to aqueous fluid and therapeutic molecules.

According to an aspect of the present invention, there is provided a synthetic polypeptide sequence of a mechanosensitive channel of large conductance 1 (MscL1) protein and its generated site-directed mutant(s) thereof, wherein said MscL1 or mutant(s) thereof comprises at least 75% sequence identity to SEQ ID NO:1 and wherein the MscL1 protein or mutant(s) thereof, when expressed on mammalian cell membrane, senses pressure changes and modulates the intra-cellular pressure, or molecular transport including aqueous fluid and therapeutic molecules.

In another embodiment of the invention, the present invention describes a protein, wherein one or more of a single or combination of mutations modulate pressure sensitivity, pore size, gating, or kinetics. For example, present invention demonstrates I to L substitution at an amino acid residue corresponding to amino acid I at 113 position of the MscL sequence SEQ ID No. 1 to sensitize the cells toward ultrasound activation at very low pressure. Furthermore, hypersensitivity to stretch forces (activated at far below the threshold for MscS), increase in pressure sensitivity is achieved by G to Any other 19 naturally occurring amino acid substitution at an amino acid residue corresponding to amino acid G at 43 position of the MscL sequence ID No. 1.

In another embodiment, the present invention describes a protein, wherein to increase the pressure sensitivity and shorten the open times, V to C amino acid substitution at an amino acid residue corresponding to amino acid V at 44 position of the MscL sequence ID No. 1 is carried out. Increases in the pressure sensitivity and shorter open times is also achieved by deletion of amino acid(s) from 131 to 133, 131 to 136 or 131 to 157 position(s) of the MscL sequence ID No 1.

In another embodiment of the invention, present invention demonstrates increased pressure sensitivity of the protein (sequence ID No. 1) by substitution of K at 122 position to any negatively charged amino acid at an amino acid residue. Furthermore, substitution of K at 52 position of the MscL sequence ID No. 1 leads to increased pressure sensitivity and shorter mean open times, lower transition barrier. Q to C or P or F or W or Y or H amino acid substitution at an amino acid residue corresponding to amino acid Q at 77 position of the MscL sequence ID No. 1 leads to increased pressure sensitivity and increased mean open time, high transition barrier. In addition, L to C amino acid substitution at an amino acid residue corresponding to amino acid L at 40 position of the MscL sequence ID No. 1 results in sensitiveness to higher gating threshold.

In another embodiment of the invention, the present invention describes a synthetic polypeptide sequence of MscL2 protein (SEQ ID NO: 2) and its generated site-directed mutants The MscL2 protein when expressed on mammalian cell membrane, senses pressure changes and modulate the intra-cellular pressure, or molecular transport not limited to aqueous fluid and therapeutic molecules.

In another embodiment of the invention, the present invention describes a synthetic polypeptide sequence of MscL3 protein and its generated site-directed mutants. The MscL3 protein (SEQ ID NO: 3) that, when expressed on mammalian cell membrane, senses pressure changes and modulate the intra-cellular pressure, or molecular transport not limited to aqueous fluid and therapeutic molecules.

In another embodiment of the invention, the present invention describes a synthetic polypeptide sequence of MscL4 protein and its generated site-directed mutants comprising: An MscL4 protein (SEQ ID NO: 4) that, when expressed on mammalian cell membrane, senses pressure changes and modulate the intra-cellular pressure, or molecular transport not limited to aqueous fluid and therapeutic molecules.

It may be that said mutant(s) has the function to modulate pressure sensitivity, pore size, gating, or kinetics and is selected from one or more substitutions, deletions or combinations thereof.

In another embodiment of the invention, the present invention describes a synthetic polypeptide sequence wherein one or more of MscL protein(s) or chimera of sequence elements of different MscL protein(s) or concatemers of MscL monomers expressed in a cell is fused to one or more amino acid sequence motifs selected from the group consisting of a signal peptide, an endoplasmic reticulum (ER) export signal, a membrane trafficking signal, and an N-terminal Golgi export signal. Specifically, the signal consists of SEQ ID NO: 7 (MLPQQVGFVCAVLALVCCASG). The signaling sequence can also be selected from SEQ ID NO: 8 (MGRLLALVVGAALVSSAC) or SEQ ID NO: 9 (MAVPARTCGASRPGPART) or any signaling peptide sequences.

In another embodiment of the invention, the present invention describes a method wherein heterologously expressed mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants are used as a tension activated pressure release valve in cells including trabeculocytes, epithelial cells and other cells of the body that are subjected to mechanical or osmotic pressure for therapeutic purpose.

In another embodiment of the invention, the present invention describes a method and compositions, wherein the MscL homologues strains include but not limited to bacterial, fungi, yeast etc.

In yet another embodiment, the present invention describes the compositions, wherein the Mscl homologues strains includes but not limited to MscLs from Escherichia coli, Mycobacterium tuberculosis, Vibrio cholera, Bacillus subtilis, Mycobacterium leprae, Chlorobium tepidum, Thermus thermophilus, Haemophilus influenza, Erwinia carotovora, Pseudomonas fluorescens, Clostridium perfringens, Staphylococcus aureus, Streptococcus faecalis, Lactococus lactis, Brucella melitensis, Caulobacter crescentus, Clostridium histolyticum, Fusobacterium nucleatum subsp, Mesorhizobium loti, Pasteurella multocida, Pectobacterium carotovorum, Pseudomonas aeruginosa, Salmonella enterica serovar Typhimurium, Salmonella enterica serovar Typhi, Xylella fastidiosa, Corynebacterium glutamicum, Deinococcus radiodurans, Lactococcus lactis, Ralstonia solanacearum, Sinorhizobium meliloti, Streptococcus pneumoniae, Streptococcus pyogenes, Streptomyces coelicolor, Methanosarcina acetivorans Listeria innocua, Listeria monocytogenes etc.

In another embodiment, this invention provides a method wherein MscL would function as an alternative outflow actuator for the treatment of POAG.

In another embodiment, this invention provides a method, wherein MscL function would supplement native paracytosis and transcytosis in the movement of aqueous humor around and through the endothelial cells of the TM, thereby alleviating outflow resistance and lowering of IOP for the treatment of POAG (Primary open angle glaucoma).

In yet another embodiment of the invention, the present invention describes a method wherein the said mechanosensitive channel (e.g., MscL) is delivered through viral vectors (e.g., adeno virus, adeno associated-virus, lentivirus) or non-viral methods and said channel acts as a transgenic pressure modulator in the impaired cells of glaucomatous eyes in selected but not limited to Trabecular Meshwork (TM) cells by use of promoters such as matrix Gla protein (MGP) promoter.

In another embodiment of the invention, the present invention describes non-viral methods (lipofection, nanoparticle/laser-mediated delivery) for delivery of the mechanosensitive channel encoding genes to the targeted tissue.

Another aspect of this invention describes the application of MscL as an ideal candidate to use as a drainage valve in trabeculocytes because it is a relatively small homo-oligomeric channel and does not need any associated proteins or energy sources to assemble and function.

In yet another embodiment, this invention provides a method wherein, the said mechanosensitive channel, its variants, and the generated site-directed mutants are activated at selected but not limited to physiological and non-physiological pressures such as greater than 20 mmHg in eye; or 120-200 mmHg (systolic) and 80-110 (diastolic) in artery; or greater than 15 mmHg intracranial pressure.

In a specific embodiment, this invention demonstrates delivery and expression of MscL in the cells of trabecular meshwork (TM). The MscL microbial channel would function in the foreign environment of the TM and lead towards lowering of IOP in glaucoma patients.

In yet another embodiment, this invention provides a method wherein the expression of the said mechanosensitive channel in meibomian glands endothelial cells, corneal epithelial cells etc with or without mechanical stimulation would lead to enhanced secretion of aqueous phase of the tear film and thus, alleviation of dry eye disease (DED).

In yet another embodiment, this invention provides a method wherein, MscL, or its variants, or generated site-directed mutants, when expressed in mammalian cells, can be used for molecular delivery into the cells by application of extra-cellular stimuli including osmotic stress or other mechanical actuation not limiting to ultrasound and hydrodynamic pressure.

In yet another embodiment, this invention provides a method wherein, MscL, or its variants, or generated site-directed mutants, when expressed in mammalian cells, can be used for stimulation of the cells by application of extra-cellular stimuli not limiting to ultrasound modulation.

In a broader embodiment, this invention provides a composition and method wherein MscL or its variants, or generated site-directed mutants, delivered via viral or non-viral (physical, chemical) method, expressed in targeted mammalian cells in a promoter-specific manner, the microbial channel(s) would function in the foreign environment under specific range of mechanical stimuli or osmotic stress generated inside the body or externally using specific device not limiting to ultrasound modulation.

It is contemplated that any embodiment of a method, device or composition described herein can be implemented with respect to any other method, device or composition described herein.

In one aspect, the amino acid has at least one of 75%, 85%, 95% or 100% identity to SEQ ID NO: 1, 2, 3, 4, 5 or 6.

In yet another embodiment of the invention, the present invention describes a method wherein the mammalian cells expressing the mechanosensitive channels such as bacterial mechanosensitive channels (MscL, MscS, MscK, MscG), plant mechanosensitive channels (MSL2-10), TRPV1-TRPV5 channels, Piezo channels, or their generated site-directed mutants, have a role in controlling hypertension in organs.

In yet another embodiment of the invention, the present invention describes a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used as a tension activated pressure release valve in brain cells such as arachnoid granulations (specifically the arachnoid cap cells) for reducing intracranial hypertension.

In an embodiment of the invention, the present invention describes a method wherein the endothelial cells (For example, endothelial cells of the glomerulus or the excretion apparatus in the kidney) expressing the mechanosensitive channels such as bacterial mechanosensitive channels (MscL, MscS, MscK, MscG), plant mechanosensitive channels (MSL2-10), TRPV1-TRPV5 channels, Piezo channels, or their generated site-directed mutants, have a role in controlling hypertension in organs.

In an embodiment of the invention, the present invention describes a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels, or their generated site-directed mutants derived from alternative strains are delivered locally, intraocular, intravenous, intrathecal, intramuscular or subcapsular.

In yet another embodiment of the invention, the present invention describes a method wherein the mammalian expressing the mechanosensitive channels such as bacterial mechanosensitive channels (MscL, MscS, MscK, MscG), plant mechanosensitive channels (MSL2-10), TRPV1-TRPV5 channels, Piezo channels or their generated site-directed mutants have neuroprotective effects on the cells.

In another embodiment of the invention, the present invention describes a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used for improving viability and functioning of cells including neurons, endothelial, epithelial, muscular, cardiac cells.

In another embodiment of the invention, the present invention describes a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used for neuromodulation in conjunction with external devices such as ultrasound, photoacoustic or other physical methods for direct or indirect pressure-modulation.

In yet another embodiment of the invention, the present invention describes a method wherein the mammalian cells expressing the mechanosensitive channels such as bacterial mechanosensitive channels (MscL, MscS, MscK, MscG), plant mechanosensitive channels (MSL2-10), TRPV1-TRPV5 channels, Piezo channels or their generated site-directed mutants, have a role in regulating fibroproliferative activity. In yet another embodiment of the invention, the present invention describes a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used for therapeutic use without eliciting immune response.

In yet another embodiment of the invention, the present invention describes a method wherein the cells in anterior cortex chamber (ACC) expressing the mechanosensitive channels such as bacterial mechanosensitive channels (MscL, MscS, MscK, MscG), plant mechanosensitive channels (MSL2-10), TRPV1-TRPV5 channels, Piezo channels or their generated site-directed mutants reduce pain.

In yet another embodiment of the invention, the present invention describes a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used as a tension activated pressure release valve in cells in diseases characterized by elevated pressure or impaired permeability of tissues.

Details associated with the embodiments described above and others are described below.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The following drawings illustrate by way of example and not limitation. For the sake of brevity and clarity, every feature of a given structure is not always labeled in every figure in which that structure appears. The following drawings form part of the present specification and are included to further demonstrate certain aspects of the present invention. The invention may be better understood by reference to one or more of these drawings in combination with the detailed description of the specification embodiments presented herein.

The patent or application file contains at least one drawing executed in color. Copies of this patent or patent application publication with color drawing(s) will be provided by the Office upon request and payment of the necessary fee.

Tables 1-6 show Amino acid sequences of different MscLs: MscL1, MscL2, MscL3, and MscL4. Different promoters (e.g., pMGP, CAG) are used upstream of MscL-sequences for targeting specific cells as an example.

FIG. 1A. Typical domain architecture of MscL gene constructs with 5′ promoter sequences and 3′ fluorescent (mCherry) reporter sequences. FIG. 1B. Typical circular DNA plasmid map showing the insertion of MscL gene construct cloned between two restriction sites (BamH I and Sal I).

FIG. 2A. Bright field image of HEK293 cells transfected with plasmid DNA containing the EcMscL-mCherry gene. FIG. 2B. Red filtered confocal fluorescence image of HEK293 cells transfected with plasmid DNA containing the EcMscL-mCherry gene. 488 nm excitation and 560-660 nm emission filter. FIG. 2C. Stable current profile of an EcMscL expressing HEK293 cell before hypotonic shock. FIG. 2D. Inward current profile of an EcMscL expressing HEK293 cell in response to hypotonic shock (addition of 20% v/v water).

FIG. 3A. Composite of bright field and red filtered confocal fluorescence images of embryonic rat trabecular meshwork cells transfected with plasmid DNA containing the EcMscL-mCherry gene. FIG. 3B. Zoomed confocal fluorescence (560-660 nm emission) image of two embryonic rat trabecular meshwork cells transfected with plasmid DNA containing the EcMscL-mCherry gene. FIG. 3C. Inward current profile of an EcMscL expressing rat TM cell in response to hypotonic shock (addition of ˜20$% v/v water). FIG. 3D. Comparison of EcMscL channel peak current and open dwell times in TM and HEK cells. N=5, AV±S.D. *p<0.001.

FIG. 4A. Bright field image of embryonic rat trabecular meshwork cells transfected with plasmid DNA containing the EcMscL-mCherry gene. FIG. 4B. Confocal fluorescence (560-660 nm emission) image of embryonic rat trabecular meshwork cells transfected with plasmid DNA containing the EcMscL-mCherry gene. FIG. 4C. Confocal fluorescence (488 nm excitation and 505-525 nm emission) image of embryonic rat trabecular meshwork cells transfected with plasmid DNA containing the EcMscL-mCherry gene. FIG. 4D. Bright field image of embryonic rat trabecular meshwork cells, transfected with plasmid DNA containing the EcMscL-mCherry gene, in ˜1 U/mL Alexa Fluor™ 488 Phalloidin dye. FIG. 4E. Confocal fluorescence (488 nm excitation and 505-525 nm emission) image of embryonic rat trabecular meshwork cells, transfected with plasmid DNA containing the EcMscL-mCherry gene, in ˜1 U/mL Alexa Fluor™ 488 Phalloidin dye.

FIG. 4F. Confocal fluorescence (488 nm excitation and 505-525 nm emission) image of embryonic rat trabecular meshwork cells, transfected with plasmid DNA containing the EcMscL-mCherry gene, in ˜2 U/mL Alexa Fluor™ 488 Phalloidin dye. FIG. 4G. Bright field image of embryonic rat trabecular meshwork cells, transfected with plasmid DNA containing the EcMscL-mCherry gene, in ˜2 U/mL Alexa Fluor™ 488 Phalloidin dye. 15 minutes after a hypo-osmotic shock (addition of 20% v/v water). The control (not expressing EcMscL-mCherry) is marked by arrow. FIG. 4H. Confocal fluorescence (488 nm excitation and 505-525 nm emission) image of embryonic rat trabecular meshwork cells, transfected with plasmid DNA containing the EcMscL-mCherry gene, in ˜2 U/mL Alexa Fluor™ 488 Phalloidin dye. 7.5 minutes after a hypo-osmotic shock (addition of 20% v/v water). 488 nm excitation and 505-525 nm emission filter. FIG. 4I. Green filtered confocal fluorescence image of embryonic rat trabecular meshwork cells, transfected with plasmid DNA containing the EcMscL-mCherry gene, in ˜2 U/mL Alexa Fluor™ 488 Phalloidin dye. 15 minutes after a hypo-osmotic shock (addition of 20% v/v water). 488 nm excitation and 505-525 nm emission filter. FIG. 4J. Kinetics of the intensity of green fluorescence in cells, showing only EcMscL expressing cells uptake the dye, while non transfected cells stay dark.

FIG. 5A. Principle of the response of cells sensitized with MscL to membrane tension/deformation induced by mechanical force stimuli. FIG. 5B. Principle of controlling intraocular pressure using MscL sensitization of trabecular meshwork cells.

The MGP-EcMscL-mCherry gene packaged in AAV8 virus and delivered to anterior chamber of mice eyes via intracameral injection led to expression in trabecular meshwork (TM). FIG. 6A. Confocal DAPI stained (blue fluorescence) image of Irido-corneal region of a cross-section of an eye treated with MGP-EcMscL-mCherry gene packaged in AAV8 virus showing the cells in cornea and TM. FIG. 6B. Confocal anti-mCherry antibody stained (red fluorescence) of the cross-section of the eye treated with MGP-EcMscL-mCherry gene packaged in AAV8 virus. The strong red fluorescence of reporter (mCherry) seen in the TM shows successful targeted expression of EcMscL-mCherry in the TM. FIG. 6C. Elevated IOP in mice treated with topical application of 0.1% dexamethasone (DEX) 3 times daily. (N=7). FIG. 6D. Decrease in intraocular pressure (IOP) of OD eye 3 weeks after intracameral injection of virally carried EcMscL into OD eye targeted to the TM. (N=4). Average±Std. Dev. FIG. 6E. IOP decrease in contralateral (OS) eye 3 weeks after intracameral injection of virally carried EcMscL in the OD eye. (N=4). Average±Std. Dev.

FIG. 7. Confocal Image of the flat-mount mouse retina transfected with EcMscL-double mutant (I113L/I70E, SEQ ID No. 5) via intravitreal injection and nano-enhanced optical delivery (NOD), a type of non-viral (laser assisted) gene delivery. As laser irradiation was done in the rectangular region (marked by dotted boundary), the anti-mCherry antibody immune-stained retina shows red fluorescence in transfected area.

The MGP-EcMscL(SEQ ID NO. 1)-mCherry gene packaged in AAV8 virus and delivered via lateral tail vein injection to a mice led to expression in brain regions. FIG. 8A. DAPI stained confocal (blue fluorescence) image of the brain section of a mouse transfected with EcMscL-mCherry in the cerebellum region. The scale bar is 20 micron. FIG. 8B. Confocal anti-mCherry antibody stained (red fluorescence) image showing transfection and EcMscL expression at the edge of cerebellum. The arrows show transfected regions. FIG. 8C. DAPI stained confocal (blue fluorescence) image of the brain section of a mouse transfected with EcMscL-mCherry in the Pons and Medulla region. FIG. 8D. Confocal anti-mCherry antibody stained (red fluorescence) image showing transfection and EcMscL expression in the Pons and Medulla region. The arrows show transfected regions.

FIG. 9A. Confocal image of reporter (mCherry) fluorescence transfected HEK293 cells expressing MtMscL-mCherry (see SEQ ID NO. 2) showing plasma membrane localization of the protein expression. FIG. 9B. Inward current profile of a patched MtMscL expressing HEK293 cell after hypotonic shock (addition of 20% v/v water). FIG. 9C. Overlay of red and green filtered confocal images of HEK293 cells transfected with the MtMscL-mCherry gene in solution containing the membrane impermeable dye (Alexa Fluor™ 488 carboxylic acid succinimidyl ester, green) 25 minutes after hypotonic shock. The image shows uptake of the dye (green fluorescence) only in MtMscL expressing cells. FIG. 9D. Kinetics of the intensity of green fluorescence in cells showing MtMscL expressing cells (e.g., Cell 1, Cell 2, Cell 3) uptake the dye, while non-transfected cells stay dark.

FIG. 10A. Confocal image of HEK293 cells transfected with the EcMscL(I113L/I70E)-mCherry gene (see SEQ ID NO. 5) in solution containing the membrane impermeable dye, Alexa Fluor™ 488 carboxylic acid succinimidyl ester. FIG. 10B. Inward current profile of a patched EcMscL(I113L/I70E) expressing HEK293 cell in response to a hypotonic shock (addition of 20% v/v water). FIG. 10C. Kinetics of the intensity of green fluorescence of the membrane impermeable dye (Alexa Fluor™ 488 carboxylic acid succinimidyl ester) in cells, showing only EcMscL(I113L/I70E) expressing cells (e.g., Cell 1, Cell 2, Cell 3) uptake the dye, while non-transfected cells stay dark.

FIG. 11A. Overlay of red and green filtered confocal images of HEK293 cells transfected with the VcMscL-mCherry gene (see SEQ ID NO. 4) in solution containing the membrane impermeable dye, Alexa Fluor™ 488 carboxylic acid succinimidyl ester. FIG. 11B. Kinetics of the intensity of green fluorescence in cells, showing only VcMscL expressing cells (e.g., Cell 1, Cell 2, Cell 3 and Cell 4) uptake the membrane impermeable dye, Alexa Fluor™ 488 carboxylic acid succinimidyl ester, while non-transfected cells stay dark.

FIG. 12A. Confocal Image of anti-mCherry antibody immune-stained blood vessels in mouse. FIG. 12B. Confocal Image of anti-mCherry antibody immune-stained blood vessels in mouse transfected with EcMscL-double mutant (I113L/I70E, SEQ ID No. 5) showing red fluorescence in the walls of blood vessels.

FIG. 13. Extracellular potential of EcMscL expressing HEK293 cells grown on a multi-electrode array (MEA) petri dish in response to hypotonic shock (addition of 20% v/v water). Burst of spikes can be seen in the signal during 13-14.5 sec, demonstrating robust EcMscL channel activities.

FIG. 14A. Extracellular potential of HEK293 cells (−ve control) grown on a multi-electrode array (MEA) petri dish. FIG. 14B. Ultrasound stimulation (Pulse width: 250 ms, Repetition rate: 2 Hz, Frequency: 1.1 MHz) induced EcMscL channel activities in EcMscL expressing HEK293 cells measured by extracellular potential using a multi-electrode array (MEA) petri dish.

FIG. 15. Confocal Image of the mouse cornea transfected with EcMscL-mCherry via AAV8 based gene transduction. Expression of reporter-mCherry exhibited by red fluorescence.

FIG. 16A. Confocal Image of the mouse Heart transfected with EcMscL-mCherry via AAV based gene transduction. Expression of reporter-mCherry exhibited by red fluorescence. FIG. 16B. Confocal Image of the mouse Kidney transfected with EcMscL-mCherry via AAV based gene transduction. Expression of reporter-mCherry exhibited by red fluorescence. FIG. 16C. Confocal Image of the mouse Liver transfected with EcMscL-mCherry via AAV based gene transduction. Expression of reporter-mCherry exhibited by red fluorescence. FIG. 16D. Confocal Image of the mouse Lung transfected with EcMscL-mCherry via AAV based gene transduction. Expression of reporter-mCherry exhibited by red fluorescence.

FIG. 17A. Fluorescence (mCherry-reporter) image of HEK cells expressing EcMscL^{m}. Scale bar: 100 mm. FIG. 17B. Inward current profile of EcMscL^{m} expressing cell subjected to −15 mmHg and −30 mmHg. Inset shows current profile of non-transfected cell subjected to −30 mmHg. FIG. 17C. Quantitative comparison of inward current for 2 different hold-pressures as compared to no pressure control in EcMscL^{m}-transfected cell, and −ve control (non-transfected) cell. N=4, Av.±S.D., ****p<0.0001. FIG. 17D. EcMscL^{m}-channel activity to sharp (˜50 ms) pressure changes.

FIG. 18A. Representative fluorescence images of flat mount anterior segment showing EcMscL^{m}-mCherry (intrinsic) expression in iridocorneal junction in mice 4 weeks post intracameral AAV-EcMscL^{m}-mCherry (vEcMscL^{m}) injection. FIG. 18B. Representative fluorescence images of flat mount anterior segment showing no red fluorescence in iridocorneal junction in DEX-only control mice. FIG. 18C. The bar plot showing intensity of mCherry-reporter signal in the TM for AAV-EcMscL^{m}-mCherry-treated and control eyes. N=5, Av±SD. ****p<0.0001. FIG. 18D. Fluorescence images of anterior chamber flat mount immunostained with mCherry (red, reporter for EcMscL^{m}) for AAV-EcMscL^{m}-mCherry-treated mouse. FIG. 18E. Fluorescence images of anterior chamber flat mount immunostained with Caspase-3 (green) for AAV-EcMscL^{m}-mCherry-treated DEX-mouse. No detectable apoptotic cells in vEcMscL^{m} treated mice. FIG. 18F. Fluorescence images of anterior chamber flat mount immunostained with Caspase-3 (green) for control wild type −ve control (PBS injected) mouse. FIG. 18G. Myocilin (TM-marker) in the Iridocorneal regions in the axial sections of eye transduced by vEcMscL^{m}. FIG. 18H. mCherry-reporter in the Iridocorneal regions in the axial sections of eye transduced by vEcMscL^{m}. FIG. 18I. Western Blot confirming EcMscL^{m} (estimated MW=˜42 kDa) expression in mice anterior chamber lysate.

FIG. 19A. The anterior chamber of the mouse eye is cannulated by using a 32-gauge steel needle, which was connected to a flow-through pressure transducer for the continuous determination of pressure within the system. FIG. 19B. Measured outflow in DEX-treated control vs. intracameral AAV-EcMscL^{m}-mCherry (vEcMscL^{m})-injected DEX-mice, Av±SD, *p<0.05.

FIG. 20A. Representative visually evoked potential (VEP) in DEX-mice after vehicle injection without intracameral injection of AAV-EcMscL^{m}-mCherry (vEcMscL^{m}). Light stimulation at 0 ms. FIG. 20B. Representative visually evoked potential (VEP) in DEX-mice 5 weeks after intracameral injection of AAV-EcMscL^{m}-mCherry (vEcMscL^{m}). Light stimulation at 0 ms. FIG. 20C. Quantitative comparison of negative VEP-amplitude in DEX-control vs. vEcMscL^{m} injected DEX-mice. N=4. Av±SD. *p<0.05. FIG. 20D. The mean difference of VEP amplitude between control and vEcMscL^{m} transduced DEX-mice shown as Gardner-Altman estimation plot. The curve indicates the resampled distribution of the mean difference, given the observed data. The mean difference was plotted on floating axes on the right as a bootstrap sampling distribution. The mean difference is depicted as a dot; the 95% confidence interval is indicated by the ends of the vertical error bar. FIG. 20E. RBPMS (RGC marker) immunostained fluorescence images in peripheral and central retina of DEX-treated mice eyes injected with DEX-only and vEcMscL^{m} treated (following 4 weeks of intracameral injection in post-DEX C57BL/6J mice). FIG. 20F. Quantification of RGC counts per mm² of the retina of DEX-control and vEcMscL^{m} transduced DEX-mice. RGCs labeled with RGC marker RBPMS, in red, counted to determine the surviving RGCs. Av±SD. *p<0.05.

FIG. 21A. AAV-EcMscL^{m}-mCherry (vEcMscL^{m})-injected mice did not exhibit immune cell response in injected organ. Immunostained (Iba1) image in vEcMscL^{m}-injected mice. FIG. 21B. Immunostained (IFNg) image of vEcMscL^{m}-injected mice shows minimal (basal) level of fluorescence implying no immune cell response in injected organ. FIG. 21C. Immunostained (Iba1) image of anterior chamber of control wild type mice. FIG. 21D. Immunostained (IFNg) image of anterior chamber of control wild type mice. FIG. 21E. Quantitative comparison of IL-6 (pro-inflammatory marker) in plasma between baseline and after 1 and 10 weeks of vEcMscL^{m} transduction. N=3 Animals, Av.±SD. No statistically significant difference implies no systemic immune response.

FIG. 22A. Non-invasive tail-cuff blood pressure monitoring by use of Volume Pressure Recording (VPR). FIG. 22B. Quantification of Systolic and diastolic blood pressure in EcMscL^{m}-mcherry (Double mutant, I113L/I70E) treated and untreated mice with elevated blood pressure (HTN mouse model). WT mouse data having normal blood pressure is included for comparison. ****P<0.0001. FIG. 22C. EcMscL^{m}-mCherry (red) immunostaining of kidney tissue of EcMscL^{m}-mCherry injected mice. FIG. 22D. mCherry (red) immunostaining of kidney tissue of non-injected (−ve control) HTN mouse. Blue: DAPI nuclei.

FIG. 23A. Using an ultrasonic probe, the mechanosensitive neurons expressing the EcMscL^{m}-mCherry in experimental mice were stimulated. Quantifiable pain responses shown during formalin assay were measured. FIG. 23B. Formalin assay scores at 5-minute intervals were reduced as compared to baselines and non-transfected sham mice in EcMscL^{m}-mCherry-sensitized treated mice. N=2, Av.±SEM.

FIG. 24. Comparison of IOP(mmHg) measurement for TGFb (fibrosis up-regulator) and Control Adeno 5 Virus models after EcMscL^{m}-mCherry expression in Trabecular Meshwork (TM) of IOP elevated mice.

FIG. 25A. In-vivo MEA recording of ultrasound stimulation evoked spiking in neurons with and without EcMscL^{m} sensitization. 25 MHz ultrasound (20 mV) stimulation induced activity in wildtype mice without EcMscL^{m} transduction (−ve control), and with EcMscL^{m} transduced neurons. FIG. 25B. Quantitative comparison of spiking rate generated in EcMscL^{m}-transduced neurons in-vivo with different intensities of ultrasound stimulation. Also shown is the ultrasound (20 mV) stimulated value from wildtype control with no EcMscL^{m} transduction. Av.±S.D., *p<0.05.

FIG. 26A. Transduction of retinal ganglion cells (RGCs) with EcMscL^{m} in rd1 retina. Reporter (mCherry) Expression (red), overlaid on Thy1-RGC marker (green). FIG. 26B. In-vivo corneal electrical recording of evoked potential in eye upon ultrasound stimulation of eye with and without EcMscL^{m} sensitization of retina. Representative profiles of 3.1 MHz ultrasound (20 mV) stimulation induced electrical activity of retina in rd1 mice without EcMscL^{m} transduction (−ve control), and with EcMscL^{m} transduced retina.

FIG. 27. Schematic showing Mechanosensitive-channel based Gene therapy for modulating pressure in pressure-related diseases associated with different organs. Insets show (limited) targets for barogenetic therapy via EcMscL^{m} expression. 1001: Glaucoma (Elevated Intraocular pressure); 1002: Stroke/Seizure (Intracranial pressure rise); 1003: Hypertensive encephalopathy (Elevated Hydrostatic force); 1004: Tinnitus (Inner ear fluid pressure); 1005: Heart Failure (Interstitial tissue pressure); 1006: Kidney disease (Interstitial Fluid accumulation); 1007: Erectile dysfunction (Blood flow abnormality); 1008: Lymphedema (Inadequate drainage of lymph fluid); 1009: Menopause (Stoppage of menstrual flow); 1010: Hypertensive cardiomyopathy (Diastolic/systolic dysfunction).

### DETAILED DESCRIPTION OF THE INVENTION

Glaucoma has been nicknamed “the sneak thief of sight”, because its onset often has no symptoms and sufferers belatedly become aware of a problem only when their vision is significantly diminished. Although glaucoma is more prevalent in the elderly, African-Americans, diabetics and those who have family members with the disease, people of all demographics are still susceptible to developing it. Glaucoma is recalcitrant to treatment and the resulting optic nerve damage is irreversible. The development of a safe effective single-dose long-lasting treatment for glaucoma would improve the lives of many glaucoma patients and revolutionize the way we treat glaucoma.

Glaucoma is defined as a group of eye conditions which cause damage to the optic nerve, usually due to elevated IOP (Intra Ocular Pressure). A healthy intraocular pressure (IOP) is maintained by continuous upkeep of vitreous and aqueous humor processing. In the case of Glaucoma patients there is a restriction in the flow of aqueous humor through the trabecular meshwork, which blocks drainage into Schlemm's canal and episcleral blood vessels, leading to elevated IOP. There are four main avenues by which IOP can be decreased; i) reduce aqueous humor production, ii) increase outflow through the uveoscleral pathway, iii) improve drainage through the conventional pathway, or iv) create an alternate outflow pathway.

Current pharmaceutical and surgical glaucoma treatments have significant limitations and side effects. The traditional pharmaceutical approach involves the delivery of a drug by daily doses of eyedrops, ointments, or oral tablets. Besides undesirable side effects, the major problem with these patient-dependent treatment protocols is poor patient compliance. To address compliance issues, researchers have begun developing non-invasive ocular implants that slowly release a drug into the eye over the period of a few months. However, many patients will still need constant adjustments to their medications and dosing regimens over time due to changes in their drug responsiveness and disease condition.

Surgical method options include, trabeculectomy, canaloplasty, laser trabeculoplasty, laser peripheral iridotomy, deep non-perforating sclerectomy, and drainage device implantation. As with all surgeries, there are risks of infection, systemic side effects and human/device failure. Post-surgical scarring at the procedure site is common and scar tissue can block or shift the newly created drainage pathway. With the added considerations of the expense, patient recovery time and inconsistent outcomes of surgery, it becomes clear that new and innovative treatments for glaucoma are needed.

As an alternative to surgical and pharmacological treatments, this invention describes a novel and unique method and compositions to treat glaucoma. The present invention provides a method to lower IOP, thereby preventing continued optic nerve damage in glaucoma patients.

It has been estimated that 80-90% of aqueous humor drainage is through the conventional outflow pathway. Studies have also elucidated that in many cases of POAG. increased IOP is associated with increased outflow resistance through the TM. Therefore it is striking to discover that few existing glaucoma surgical procedure and only one recently FDA-approved glaucoma drug, address the improvement of outflow through the conventional outflow pathway^{3,4}. Instead, they either reduce aqueous humor production (e.g. Beta-blockers) or increase uveoscleral outflow (e.g. prostaglandin analogs).

In an embodiment, this invention provides a method and composition that wherein it exploits an exogenous macromolecular pressure sensor and outflow actuator to regulate IOP by increasing outflow facility through the TM.

In another embodiment, this invention provides a method and composition, wherein exogenous macromolecular pressure sensor is the mechanosensitive channel of large conductance and its variants.

In yet another embodiment, MscL proteins of SEQ ID 1-4 and its site generated mutants when expressed on mammalian cell membrane, senses pressure changes and modulate the intra cellular pressure or molecular transport not limited to aqueous fluid and therapeutic molecules.

In another embodiment of the invention, the present invention describes a method and compositions, wherein the MscL homologues strains include but not limited to bacterial, fungi, yeast etc.

In yet another embodiment, the present invention describes the compositions, wherein the MscL homologues strains includes but not limited to MscLs from Escherichia coli, Mycobacterium tuberculosis, Vibrio cholera, Bacillus subtilis, Mycobacterium leprae, Chlorobium tepidum, Thermus thermophilus, Haemophilus influenza, Erwinia carotovora, Pseudomonas fluorescens, Clostridium perfringens, Staphylococcus aureus, Streptococcus faecalis, Lactococus lactis, Brucella melitensis, Caulobacter crescentus, Clostridium histolyticum, Fusobacterium nucleatum subsp, Mesorhizobium loti, Pasteurella multocida, Pectobacterium carotovorum, Pseudomonas aeruginosa, Salmonella enterica serovar Typhimurium, Salmonella enterica serovar Typhi, Xylella fastidiosa, Corynebacterium glutamicum, Deinococcus radiodurans, Lactococcus lactis, Ralstonia solanacearum, Sinorhizobium meliloti, Streptococcus pneumoniae, Streptococcus pyogenes, Streptomyces coelicolor, Methanosarcina acetivorans Listeria innocua, Listeria monocytogenes etc.

In yet another embodiment, the present disclosure also provides for the modification of MscL proteins expressed in a cell by the addition/Deletion, truncation, or substitution of one or more amino acid sequence motifs which enhance transport to the plasma membranes of mammalian cells. Consequently, in Some embodiments, MscL protein expressed in a cell is fused to one or more amino acid sequence motifs selected from the group consisting of a signal peptide, an endoplasmic reticulum (ER) export signal, a membrane trafficking signal, and an N-terminal Golgi export signal. The one or more amino acid sequence motifs which enhance MscL protein transport to the plasma membranes of mammalian cells can be fused to the N-terminus, the C-terminus, or to both the N- and C-terminal ends of the MscL protein.

Another aspect of this invention describes the viral delivery of a transgene encoding for the mechanosensitive channel of large conductance (MscL) to TM cells where, once translated and incorporated into the cell membrane, the channel can facilitate the movement of fluid out of the cell in response to an increase in IOP.

In an embodiment, the present invention provides a method and composition wherein a portion of the excreted cellular fluid will drain into Schlemm's canal and on into the vascular system, thereby reducing the volume of fluid in the eye. Logically, this decrease in fluid volume will result in a decrease in IOP.

Previous studies have shown that, when the contractility of TM cells is inhibited, the cells change shape, thereby creating more intercellular space and consequently improving AH outflow facility through the TM. Another aspect of this invention is that the activation of MscL will have a similar benefit, because the resulting decrease in cell size should also create more intercellular space and increase paracellular aqueous humor transport.

In another embodiment, the present invention provides a method and composition wherein cellular recovery processes caused by the shift in cellular equilibrium after MscL activation, may trigger several signaling cascades that could affect TM cell function. For example, in bacteria it has been shown that expression of heat shock proteins is upregulated after MscL gating during hypo-osmotic shock. In Parallel, it has been shown that glaucomatous TM cells expressing mutant myocilin (a cause of POAG in some patients) have increased outflow resistance, but when protein-folding chaperones (including heat shock proteins) are co-expressed, outflow facility improves. It is believed this improvement is due to the chaperones preventing mutant myocilin-induced protein aggregation, which restores function to the cells. With these two studies in mind, one can postulate that if MscL activation triggers heat-shock protein expression in mutant myocilin-induced glaucoma TM cells, then AH outflow facility should also be improved.

In yet another embodiment, the present invention provides a method and composition wherein the activation of MscL leads to fluid expulsion into Schlemm's canal, cell shrinkage and increased intercellular spaces and triggers cellular processes that may upregulate protective genes.

Another aspect of the invention describes MscL as an autonomous sensor and actuator that does not need partners or energy sources to function. MscL intrinsically senses tension in the membrane lipid bilayer and gates in direct response to this mechanical stimulation.

In a preferred embodiment, the present invention describes a method and composition wherein an Adeno-associated virus (AAV) compatible plasmid DNA construct, encoding the MscL gene and a TM-specific promoter, leads to the expression of MscL in TM cells. Preferably, Escherichia coli MscL (EcMscL), is selected because it is the most robust and well characterized MscL homolog. The MscL gene from the BL21(DE3) Escherichia coli strain, which encodes for a136 amino acid monomer, was retrieved and codon optimized for mammalian cell expression. The gene was put under the control of a TM-specific promoter. Several studies have shown that in the eye matrix Gla protein (MGP) is preferentially expressed in TM cells and its promoter sequence has been used to target gene expression. The MGP promoter sequence (pMGP) located on chromosome 12, 14886367-14885792 was identified by using the primers from Linton et al. (2005) to run an alignment search against the reverse compliment strand of human genome GRCh38.p12. The resulting 576 bp promoter sequence was placed on the 5′ end of the EcMscL gene. A fused fluorescent reporter was chosen to act as a visual marker of gene expression and membrane localization. As EcMscL is a homo-oligomer and needs to assemble into a functional complex postranslationally, a strictly monomeric fluorescent protein had to be chosen to avoid any disruptive interactions. To this end the monomeric fluorescent protein, m-Cherry, was C-terminally fused to MscL. Finally, the pAAV-MCS plasmid backbone is a salient base, because it has been reportedly used successfully in viral delivery of genes to the HTM.

In another embodiment, this invention describes a method and synthetic polypeptide sequence wherein primary cultured TM cells are transfected with MscL plasmid DNA by lipofection and protein expression and localization were assayed by epifluorescence confocal microscopy. Cells were also monitored by transmission microscopy to check for any changes to their morphology or growth cycle. Protein localization studies using anti-mCherry(reporter) antibodies was performed using western blot.

In yet another embodiment, this invention provides a method and synthetic polypeptide sequence, wherein retention of EcMscL native function in the TM cell membrane is shown through electrophysiological measurements of channel gating recordings from membrane patches excised from the surface of EcMscL expressing HTM cells. Mechanical stimulation was induced by applying suction to the patch clamp pipette to create negative pressure.

In another embodiment, the present invention provides a method and composition wherein mechanosensitive channels can be expressed in other cells of the eye for the treatment of glaucoma. The ocular cells include, but are not limited to, Schlemm's canal, retinal ganglion, glial, cilliary body, corneal, bipolar and photoreceptor cells. In this role, mechanosensitive channels can act as osmoregulators, influence the production and composition of aqueous and vitreous humor, protect compression stress, supplement neural activity, and such other functions.

In another aspect of this present invention, wherein the methods and compositions of mechanosensitive channels refers to all known mechanically/stretch activated channels and proteins, which include, but are not limited to, MscS, MscK, MscG, MSL2-MSL10, MCA, TPK, piezo channels, TRVP1-TRPV5, OSM-9, Mys1, Mys2 and MSC1.

Another aspect of the present invention describes a method and synthetic polypeptide sequence wherein heterologously expressed mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants are used as osmoregulators, activated by internal or external mechanical stimuli, in the epithelial cells of the arterial system in the treatment and prevention of hypo- as well as hypertension.

In another embodiment of the invention, the present invention describes a method and synthetic polypeptide sequence wherein the mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants, when expressed in the Hematopoietic stem cells, will generate red blood cells which act as osmoregulators and resistant to osmotic shock, in the treatment and prevention of hyper- as well as hypo-tension related diseases.

In another embodiment of the invention, the present invention describes a method and synthetic polypeptide sequence wherein heterologously expressed mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants are used as osmoregulators or diuretics, activated by internal or external mechanical stimuli, in the renal system in the treatment and prevention of kidney stones and chronic kidney disease.

Another aspect of the present invention describes a method and synthetic polypeptide sequence wherein heterologously expressed mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants are used as bile regulators, activated by internal or external mechanical stimuli, in liver, gallbladder or bile duct cells of the hepatic system in the treatment and prevention of gallstones.

Another aspect of the present invention describes a method and synthetic polypeptide sequence, wherein heterologously expressed mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants, are used as osmoregulators, activated by internal or external mechanical stimuli, and alternative outflow/inflow pathway for fluid in the treatment and prevention of edemas. These diseases include, but are not limited to, passive subretinal, cystoid macular, lymph-, peripheral, pulmonary, and pedal edemas as well as osteoarthiritis related swollen knees and joints.

In another embodiment of the invention, the present invention describes a method and synthetic polypeptide sequence wherein heterologously expressed mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants, in reproductive system are used as osmoregulators, and alternative outflow/inflow pathway for fluid, activated by internal or external mechanical stimuli, in the treatment and prevention of erectile dysfunction, vaginal dryness, benign prostatic hyperplasia and polycystic ovary syndrome.

In yet another embodiment of the invention, the present invention describes a method and synthetic polypeptide sequence wherein heterologously expressed mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants, are used as osmoregulators, activated by internal or external mechanical stimuli, and alternative outflow/inflow pathway for fluid in the treatment of any skin related diseases such as dysfunction of sweat glands etc.

In yet another embodiment of the invention, the present invention describes a method and synthetic polypeptide sequence wherein heterologously expressed mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants, are used as macromolecular pressure release valves, activated by internal or external mechanical stimuli, and alternative outflow pathway for fluid in the treatment of idiopathic intracranial hypertension.

In another embodiment of the invention, the present invention describes a method and synthetic polypeptide sequence of wherein heterologously expressed mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants are used as transmembrane ports in cells activated by internal or external mechanical stimuli for enhanced drug delivery in order to increase efficacy of the treatment for diseases including cancer.

Another aspect of the present invention describes a method and synthetic polypeptide sequence wherein the expression of said mechanosensitive channel in cancer cells, targeted by specific receptors such as EGFR, or pH environment, is used to precipitate cell death by either bioengineering a constitutively open “leaky” channel or through overstimulation of the channel.

Another aspect of the present invention describes a method and synthetic polypeptide sequence wherein heterologously expressed mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants, are used for stimulation of cells, including neurons, cardiac and muscle cells for treatment and prevention diseases including but not limited to, neurological diseases such as pain, epilepsy, stroke as well as cardiovascular diseases and muscular dystrophies.

In another embodiment of the invention, the present invention describes a method and synthetic polypeptide sequence of wherein heterologously expressed mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants, in neurons are activated by internal or external mechanical stimuli, for modulating neural activities in order to repair injury such as concussion, enhance neural regeneration, accelerated learning and memory processing.

In another embodiment of the invention, the present invention describes a method and synthetic polypeptide sequence wherein heterologously expressed mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants, in neurons are activated by internal or external mechanical stimuli, for modulating activities in dormant neurons in order to recover from coma, and persistent vegetative state.

In yet another embodiment of the invention, the present invention describes a method and synthetic polypeptide sequence wherein heterologously expressed mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants are used as osmoregulators, activated by internal or external mechanical stimuli, in the central nervous system (CNS) mediated barriers such as blood-cerebrospinal fluid (CSF) barrier, the blood brain barrier (BBB), the blood-retinal barrier and the blood-spinal cord barrier etc.

Another aspect of the present invention describes a method and synthetic polypeptide sequence wherein heterologously expressed mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants, activated by internal or external mechanical stimuli, are used as efflux ports for allowing the clearance of toxins such as Beta-Amyloid, Tau, Alpha-synuclein and PolyQ from central nervous system to the blood stream via the blood brain barrier (BBB), the blood-retinal barrier and the blood-spinal cord barrier in neurodegenerative diseases including but not limited to Alzheimer's, Parkinson's, and Huntington's disease.

Another aspect of the present invention describes a method and synthetic polypeptide sequence wherein mechanosensitive channels, such as bacterial mechanosensitive channels (MscL, MscS, MscK, MscG), plant mechanosensitive channels (MSL2-10), TRPV1-TRPV5 channels, Piezo channels or their generated site-directed mutants, heterologously expressed in Pneumocytes, activated by internal or external mechanical stimuli, are used as ports for exchange of oxygen and Carbon dioxide from alveolus to the blood capillaries and vice a versa via the Pneumocytes in lung diseases including but not limited to Black Lung Disease (Coal workers' pneumoconiosis).

In yet another embodiment of the invention, the present invention describes a method and synthetic polypeptide sequence wherein the cells expressing the mechanosensitive channels such as bacterial mechanosensitive channels (MscL, MscS, MscK, MscG), plant mechanosensitive channels (MSL2-10), TRPV1-TRPV5 channels, Piezo channels or their generated site-directed mutants, with or without fusion or conjugation of nanoprobes, are activated by a device providing external mechanical force or magnetic field, whose duration, frequency and strength can be tuned for controlled stimulation, molecular delivery or cellular death leading to the desired therapeutic outcome.

In yet another embodiment of the invention, the present invention describes a method and synthetic polypeptide sequence wherein the endothelial cells expressing the mechanosensitive channels such as bacterial mechanosensitive channels (MscL, MscS, MscK, MscG), plant mechanosensitive channels (MSL2-10), TRPV1-TRPV5 channels, Piezo channels or their generated site-directed mutants, have a role in controlling hypertension in organs.

In yet another embodiment of the invention, the present invention describes a method and synthetic polypeptide sequence wherein the mammalian cells expressing the mechanosensitive channels such as bacterial mechanosensitive channels (MscL, MscS, MscK, MscG), plant mechanosensitive channels (MSL2-10), TRPV1-TRPV5 channels, Piezo channels or their generated site-directed mutants, have a role in controlling hypertension in organs.

In yet another embodiment of the invention, the present invention describes a method and synthetic polypeptide sequence wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used as a tension activated pressure release valve in brain cells such as arachnoid granulations (specifically the arachnoid cap cells) for reducing intracranial hypertension.

In an embodiment of the invention, the present invention describes a method and synthetic polypeptide sequence wherein the endothelial cells (For example, endothelial cells of the glomerulus or the excretion apparatus in the kidney) expressing the mechanosensitive channels such as bacterial mechanosensitive channels (MscL, MscS, MscK, MscG), plant mechanosensitive channels (MSL2-10), TRPV1-TRPV5 channels, Piezo channels or their generated site-directed mutants, have a role in controlling hypertension in organs.

In yet another embodiment of the invention, the present invention describes a method and synthetic polypeptide sequence wherein the cells expressing the mechanosensitive channels such as bacterial mechanosensitive channels (MscL, MscS, MscK, MscG), plant mechanosensitive channels (MSL2-10), TRPV1-TRPV5 channels, Piezo channels or their generated site-directed mutants would have the same end physiological effect as diuretics but with the added benefit of self-regulation and permanence (as it would activate as soon as elevated blood pressure induced tension in the blood vessels reaches a pre-determined threshold).

In yet another embodiment of the invention, the present invention describes a method and synthetic polypeptide sequence wherein the tunable autoregulating activity of cells expressing the mechanosensitive channels such as bacterial mechanosensitive channels (MscL, MscS, MscK, MscG), plant mechanosensitive channels (MSL2-10), TRPV1-TRPV5 channels, Piezo channels or their generated site-directed mutants would induce an overall volume loss from the systemic vasculature, leading to a decrease in overall blood pressure.

In an embodiment of the invention, the present invention describes a method and synthetic polypeptide sequence wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains can delivered locally, intraocular, intravenous, intrathecal, intramuscular or subcapsular.

In yet another embodiment of the invention, the present invention describes a method and synthetic polypeptide sequence wherein the mammalian expressing the mechanosensitive channels such as bacterial mechanosensitive channels (MscL, MscS, MscK, MscG), plant mechanosensitive channels (MSL2-10), TRPV1-TRPV5 channels, Piezo channels or their generated site-directed mutants have neuroprotective effects on the cells.

In another embodiment of the invention, the present invention describes a method and synthetic polypeptide sequence wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used for viability and functioning of cells including neurons, endothelial, epithelial, muscular, cardiac cells.

In another embodiment of the invention, the present invention describes a method and synthetic polypeptide sequence wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, used for neuromodulation in conjunction with external devices such as ultrasound, photoacoustic or other physical methods for direct or indirect pressure-modulation.

In yet another embodiment of the invention, the present invention describes a method and synthetic polypeptide sequence wherein the mammalian cells expressing the mechanosensitive channels such as bacterial mechanosensitive channels (MscL, MscS, MscK, MscG), plant mechanosensitive channels (MSL2-10), TRPV1-TRPV5 channels, Piezo channels or their generated site-directed mutants, have a role in regulating fibroproliferative activity.

In yet another embodiment of the invention, the present invention describes a method and synthetic polypeptide sequence wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, responds to sharp pressure changes.

In another embodiment of the invention, the present invention describes a method and synthetic polypeptide sequence, wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used to provide protection of the cells.

In yet another embodiment of the invention, the present invention describes a method and synthetic polypeptide sequence, wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, does not elicit any local or systemic immune response.

In yet another embodiment of the invention, the present invention describes a method and synthetic polypeptide sequence wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, provides outflow facility.

In yet another embodiment of the invention, the present invention describes a method and synthetic polypeptide sequence wherein the cells in anterior cortex chamber (ACC) expressing the mechanosensitive channels such as bacterial mechanosensitive channels (MscL, MscS, MscK, MscG), plant mechanosensitive channels (MSL2-10), TRPV1-TRPV5 channels, Piezo channels or their generated site-directed mutants reduce pain.

In yet another embodiment of the invention, the present invention describes a method and synthetic polypeptide sequence wherein the cells in anterior cortex chamber (ACC) expressing the mechanosensitive channels such as bacterial mechanosensitive channels (MscL, MscS, MscK, MscG), plant mechanosensitive channels (MSL2-10), TRPV1-TRPV5 channels, Piezo channels or their generated site-directed mutants reduce pain generated from any kind of pressure.

In yet another embodiment of the invention, the present invention describes a method and synthetic polypeptide sequence wherein the cells in anterior cortex chamber (ACC) expressing the mechanosensitive channels such as bacterial mechanosensitive channels (MscL, MscS, MscK, MscG), plant mechanosensitive channels (MSL2-10), TRPV1-TRPV5 channels, Piezo channels or their generated site-directed mutants can sensitize the inhibitory neurons to reduce pain generated from by the pressure.

In yet another embodiment of the invention, the present invention describes a method and synthetic polypeptide sequence wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used as a tension activated pressure release valve in cells in diseases characterized by elevated pressure or impaired permeability of tissues, including but not limited to Glaucoma (Elevated Intraocular pressure), Stroke/Seizure (Intracranial pressure rise), Hypertensive encephalopathy (Elevated Hydrostatic force), Heart Failure (Interstitial tissue pressure), kidney disease (Interstitial Fluid accumulation), Erectile dysfunction (Blood flow abnormality), Lymphedema (Inadequate drainage of lymph fluid), Menopause (Stoppage of menstrual flow), and Hypertensive cardiomyopathy (Diastolic/systolic dysfunction).

The present disclosure will now be described more fully hereinafter with reference to the accompanying drawings, in which some exemplary embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

Throughout this application, the term “about” is used to indicate that a value includes the standard deviation of error for the device or method being employed to determine the value.

Further, a molecule or method that is configured in a certain way is configured in at least that way, but it can also be configured in other ways than those specifically described.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Any embodiment of any of the apparatuses, systems, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

To the extent that any specific disclosure in the aforementioned references or other literature may be considered to anticipate any generic aspect of the present invention, the disclosure of the present invention should be understood to include a proviso or provisos that exclude of disclaim any such species that were previously disclosed. The aspects of the present invention, which are not anticipated by the disclosure of such literature, are also nonobvious from the disclosure of these publications, due at least in part to the unexpectedly superior results disclosed or alleged herein.

Below, the presently disclosed invention will be further described by way of examples, which are provided for illustrative purposes only and accordingly are not to be construed as limiting the scope of the invention.

### EXAMPLES

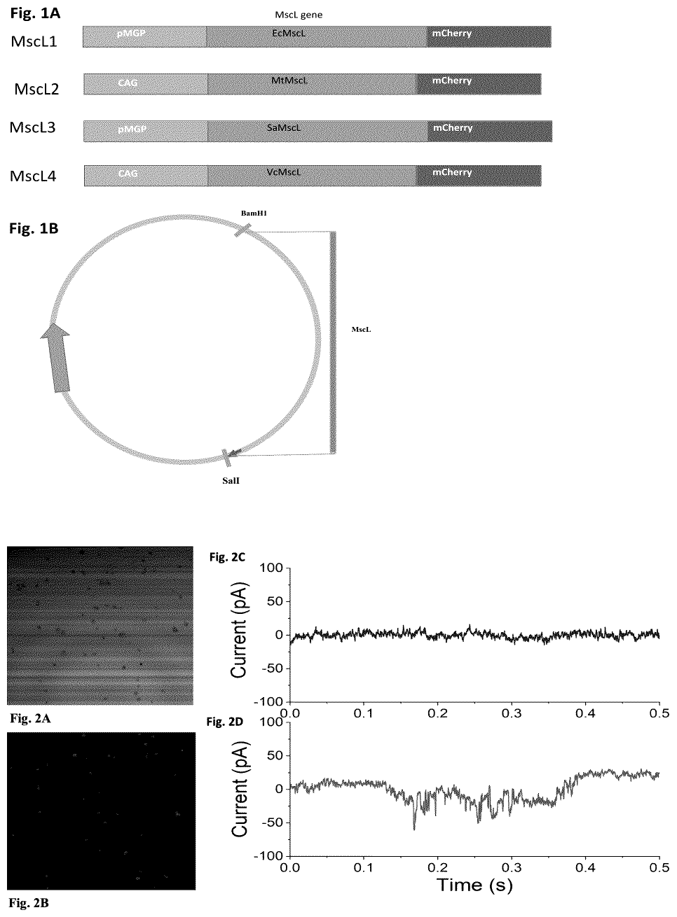

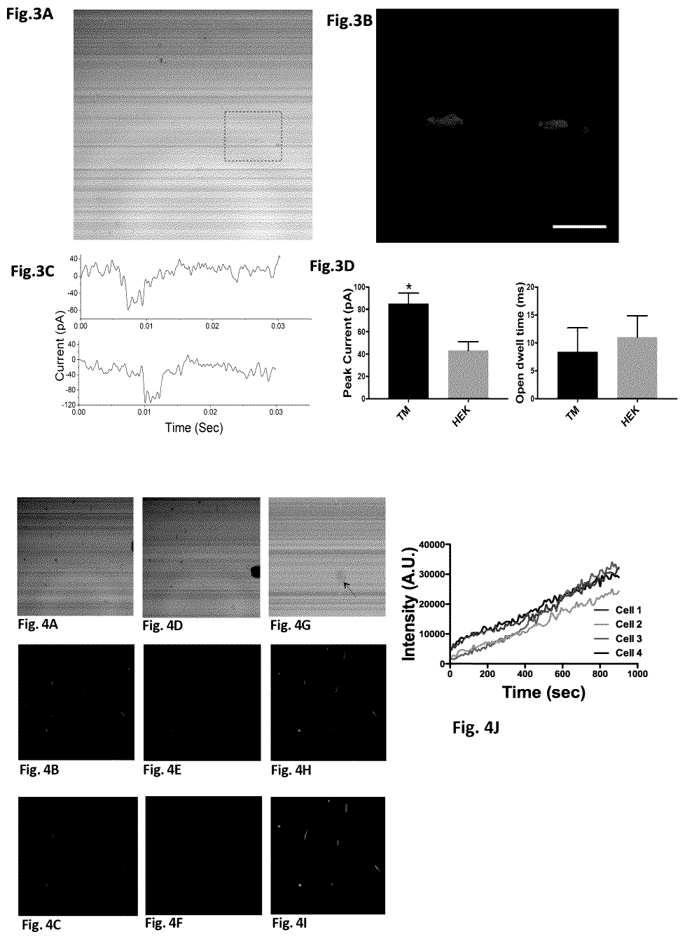

Example 1—The experiments outlined below demonstrate that bacterial mechanosensitive channels can be expressed in mammalian cells, especially trabecular meshwork cells. Escherichia coli MscL (EcMscL), has been selected as a model channel because it is the most robust and well characterized mechanosensitive channel. The MscL gene from the BL21(DE3) E. coli strain, which encodes for a 136 amino acid monomer, was codon optimized for mammalian cell expression and was put under the control of a TM-specific promoter. Several studies have shown that in the eye matrix Gla protein (MGP) is preferentially expressed in TM cells and its promoter sequence has been used to target gene expression. The MGP promoter sequence (pMGP) located on chromosome 12, 14886367-14885792 was identified by using the primers from Linton et al. (2005) to run an alignment search against the reverse compliment strand of human genome GRCh38.p12. The resulting 576 bp promoter sequence was placed on the 5′ end of the EcMscL gene. A fused fluorescent reporter was chosen to act as a visual marker of gene expression and membrane localization. As EcMscL is a homo-oligomer and needs to assemble into a functional complex post-translationally, a strictly monomeric fluorescent protein had to be chosen to avoid any disruptive interactions. To this end the monomeric fluorescent protein, m-Cherry, was C-terminally fused to MscL. This designed DNA segments were inserted into a vector between two restriction sites, BamHI and SalI [FIG. 1A and FIG. 1B]. Two mammalian cell types were transfected with the pMGP-EcMscL-mCherry construct: HEK239 cells and primary embryonic rat trabecular meshwork cells. Both cell types were cultured in 1 mL of standard DMEM growth media in 35 mm petri dishes. JetPRIME (PolyPlus) was used to deliver 2 μg of pMGP-EcMscL-mCherry plasmid DNA to each dish. Fluorescent confocal microscopy of live cells was used to confirm the successful transfection and expression of the fluorescent reporter linked gene. The samples were excited at 488 nm and a 560-660 nm red emission filter was used to visualize mCherry fluorescence. A population of HEK293 cells [FIG. 2A and FIG. 2B] and embryonic rat trabecular meshwork cells [FIG. 3A and FIG. 3B] were fluorescent, thereby indicating that MscL can be expressed in mammalian hosts that are biosimilar to the intended host (human cells).

Example 2—The experiment outlined below demonstrates that bacterial mechanosensitive channels retain their native function in mammalian cells and can be used for molecular delivery and osmotic pressure control. EcMscL is an osmoregulator and gates in response to stretching of the cell membrane caused by turgor pressure. When the EcMscL channel opens, membrane impermeable molecules are able to diffuse into and out of the cell through the open pore. Therefore, when subjected to a hypo-osmotic shock in the presence of extracellular membrane impermeable molecules (including dyes or therapeutic agents), cells expressing functional EcMscL should uptake the molecule(s). Primary cultured embryonic rat trabecular meshwork cells transfected with pMGP-EcMscL-mCherry plasmid DNA were subjected to a hypo-osmotic shock in the presence of 2 U/mL Alexa Fluor™ 488 carboxylic acid succinimidyl ester (Invitrogen) by the addition of 20% water v/v. The cells were then monitored by confocal fluorescence microscopy for 15 minutes at 488 nm excitation and detected though a green emission filter (505-525 nm). Bright field and fluorescence images show that after hypo-osmotic shock, only red (emission filter: 560-660 nm) fluorescent EcMscL expressing cells uptake the green dye [FIG. 4A to FIG. 4I]. Analysis of fluorescence images show that after hypo-osmotic shock, only red fluorescent EcMscL expressing cells uptake the green dye [FIG. 4J]. Non-fluorescent cells stay swollen and dark after the osmotic down shock indicating there was no EcMscL activity (i.e., no transport of extra-cellular impermeable dye Alexa Fluor™ 488 carboxylic acid succinimidyl ester). Therefore, we can deduce that red fluorescence is correlated with EcMscL activity and that transfection of the EcMscL-mCherry construct led to the expression of a functional membrane-integrated EcMscL-mCherry channel protein that led to osmoregulation and molecular delivery.

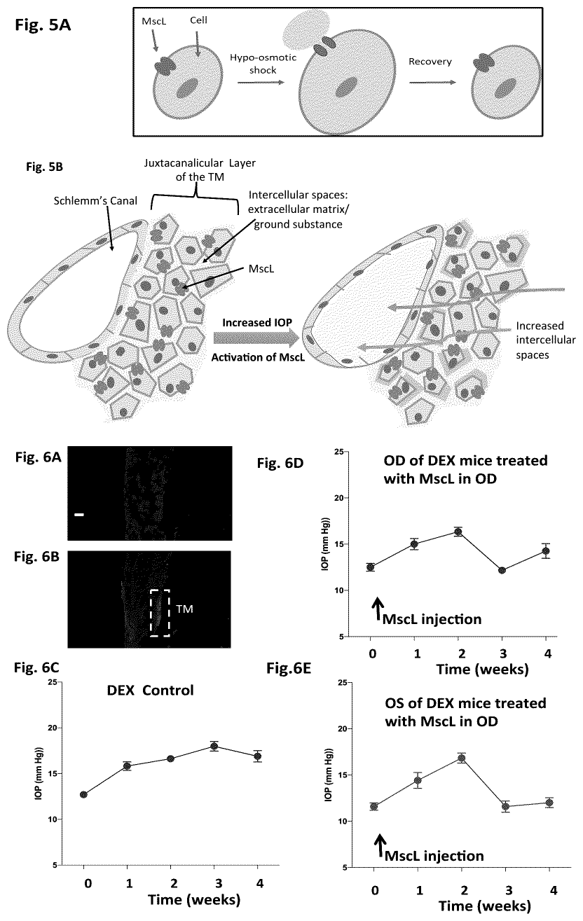

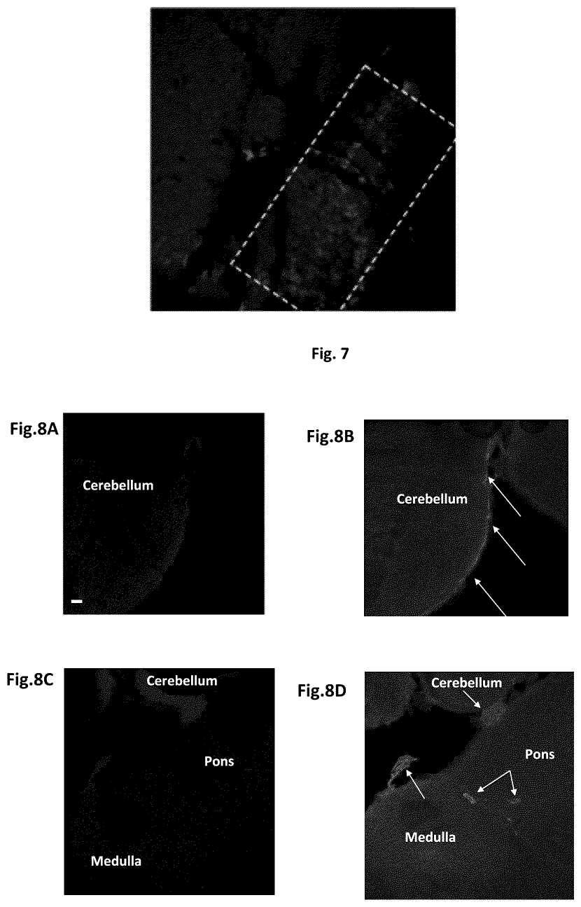

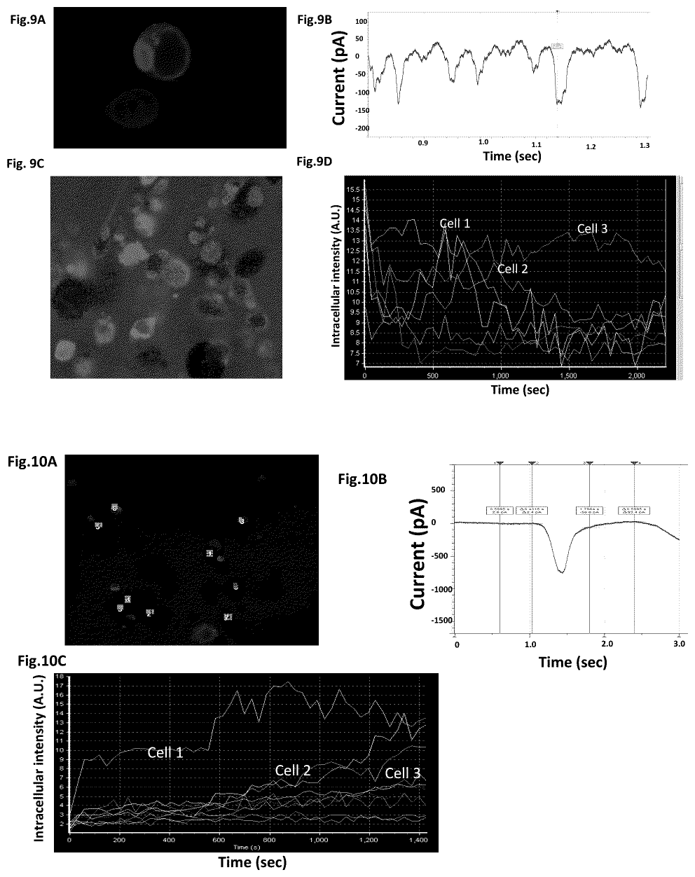

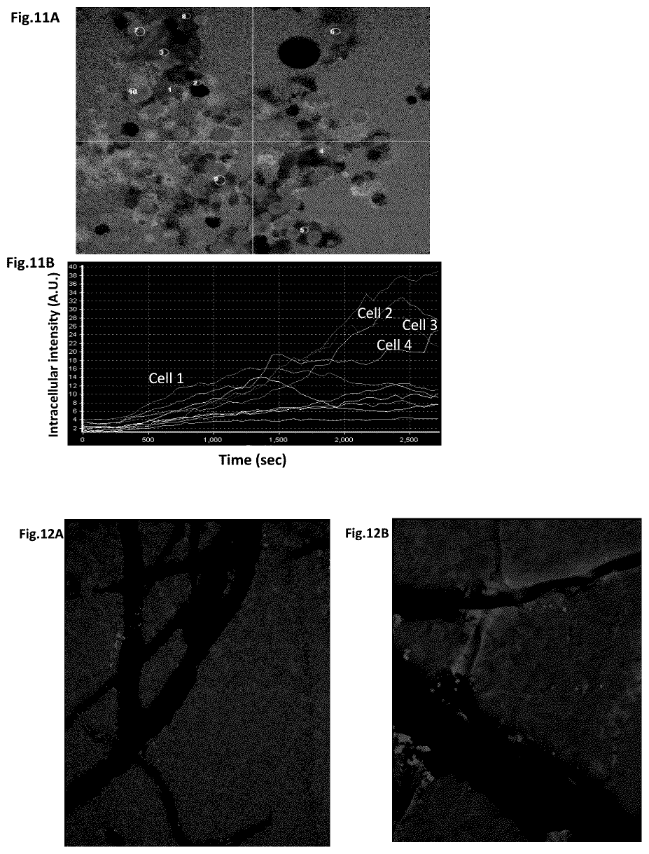

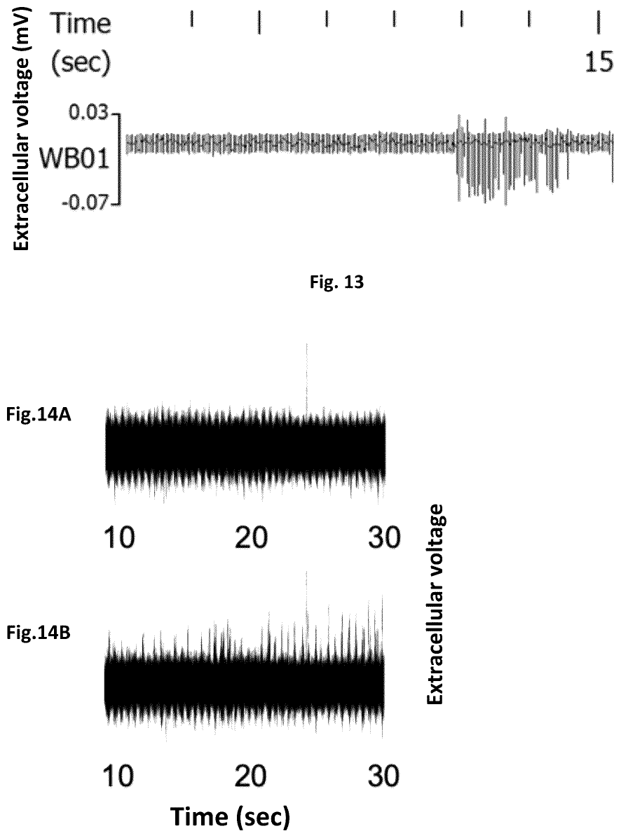

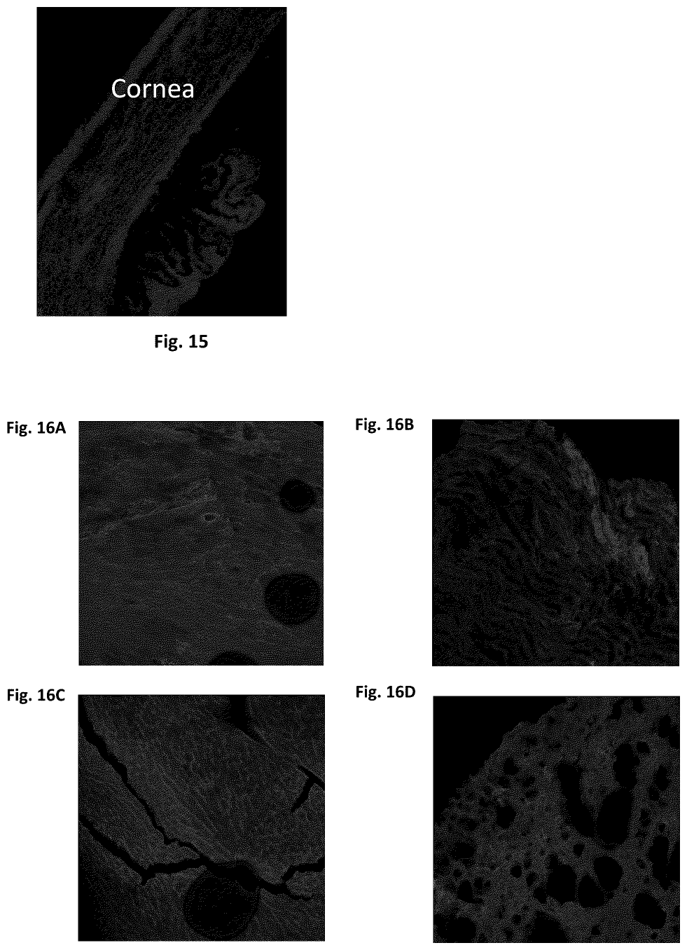

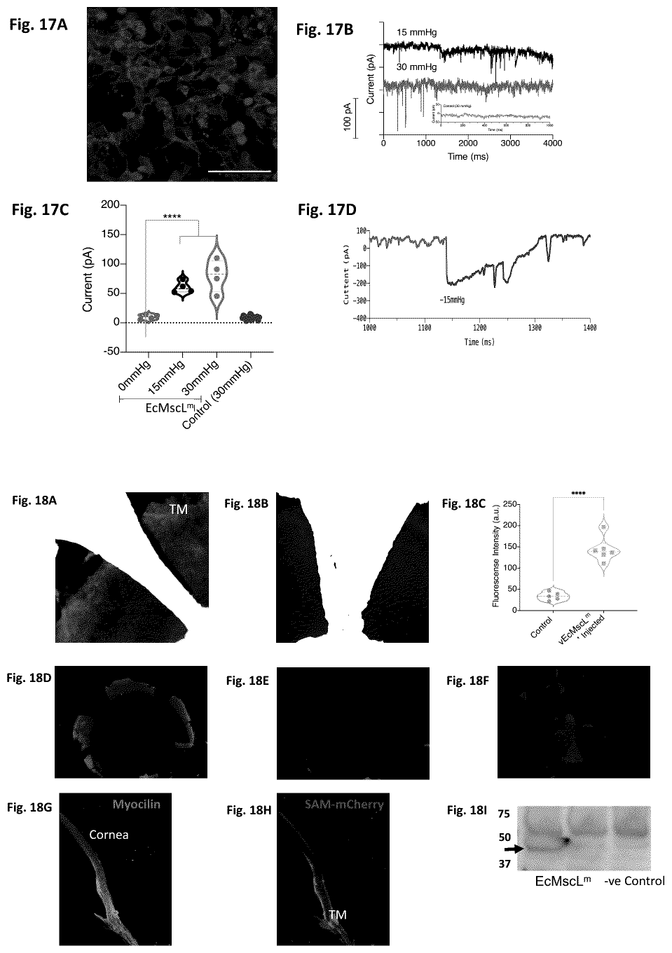

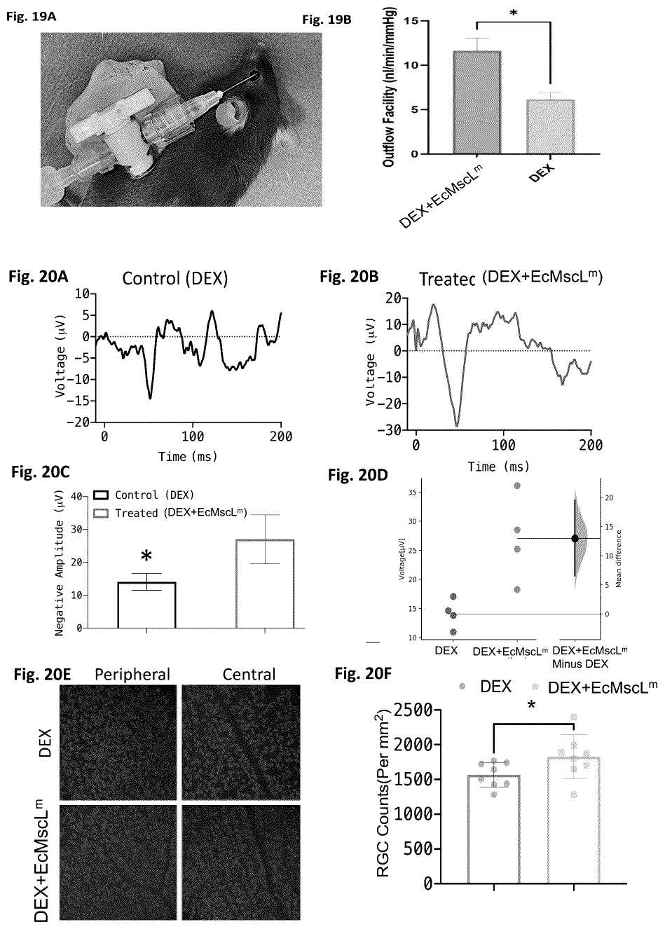

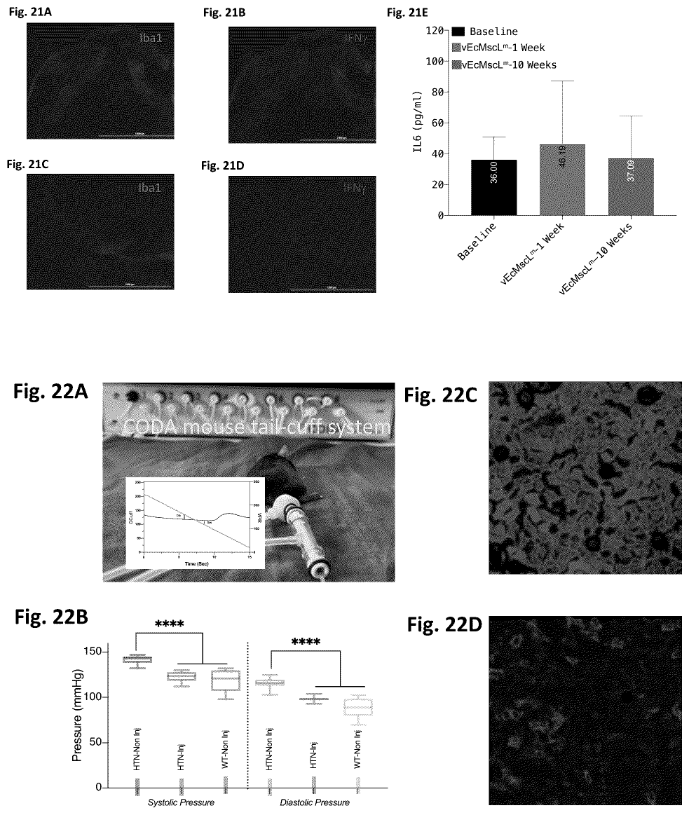

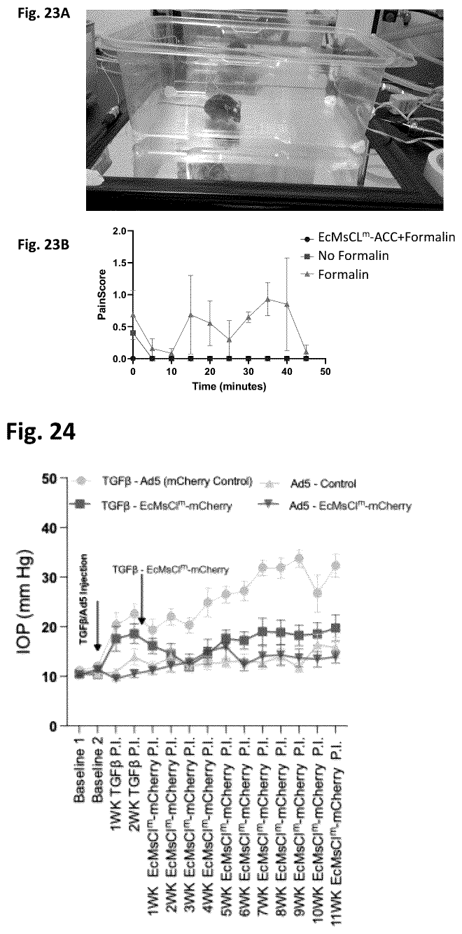

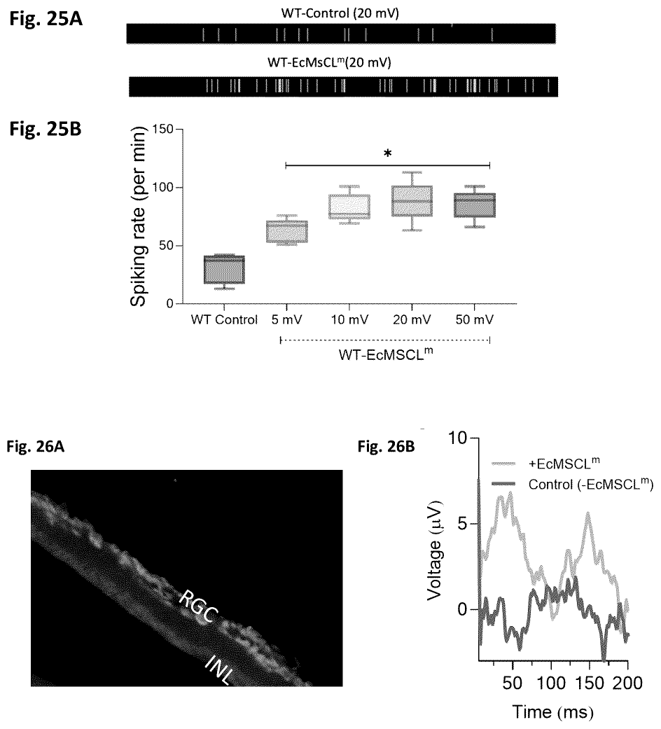

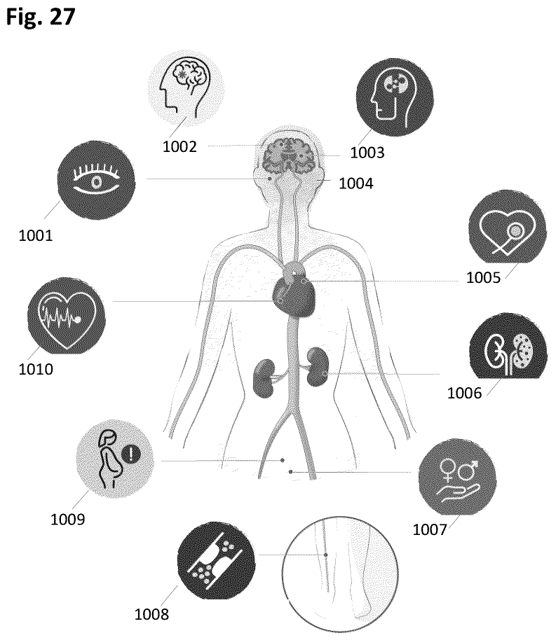

Example 3—FIG. 5A shows the principle of the response of cells sensitized with MscL to membrane tension/deformation induced by mechanical force stimuli. Principle of controlling intraocular pressure using MscL sensitization of trabecular meshwork cells is shown in FIG. 5B. The experiment outlined below demonstrates that bacterial mechanosensitive channels retain their native functional in mammalian cells and can be stimulated by external mechanical or osmotic pressure. EcMscL channel function in mammalian cells was probed by recording electrophysiological measurements from whole cell patches of HEK293 and embryonic rat TM cells expressing EcMscL-mCherry. The patch-clamp recording setup includes an inverted Nikon fluorescence microscope (TS 100) platform using an amplifier system (Axon Multiclamp 700B, Molecular Devices). Micropipettes were pulled using a two-stage pipette puller (Narshinghe) to attain resistance of 3 to 5 MΩ when filled with a solution containing (in mM) 130 K-Gluoconate, 7 KCl, 2 NaCl, 1 MgCl₂, 0.4 EGTA, 10 HEPES, 2 ATP-Mg, 0.3 GTP-Tris and 20 sucrose. The micropipette-electrode was mounted on a micromanipulator. The extracellular solution contained (in mM): 150 NaCl, 10 Glucose, 5 KCl, 2 CaCl₂), 1 MgCl₂ was buffered with 10 mM HEPES (pH 7.3). Photocurrents were measured while holding cells in voltage clamp at −70 mV. The electophysiological signals from the amplifier were digitized using Digidata 1440 (Molecular devices), interfaced with patch-clamp software (Clampex, Molecular Devices). pClamp 10 software was used for data analysis. Channel activity was induced by subjecting the patched cell to a hypo-osmotic shock by the addition of 20% v/v water [FIG. 2C and FIG. 3C]. A stable GigaOhm seal was achieved, and the current traces show channel activity in response to the hypo-osmotic shock. A comparison of channel activity in HEK293 cells and embryonic rat TM cells show a difference in peak current and open dwell times [FIG. 3D].

Example 4—The experiments outlined below demonstrate that when expressed in the trabecular meshwork (TM), EcMscL can lower intraocular pressure (IOP) and thereby treat glaucoma. Studies on the effect of virally delivered EcMscL on IOP were carried out in a glucocorticoid-induced mouse model of glaucoma. To induce elevated IOP in wild-type C576L/6J mice, 0.1% dexamethasone (DEX) ophthalmic solution was administered to the mouse eye three times daily for the duration of the study. IOP was measured using a TonoVet (a rebound tonometer). The IOP readings for each eye were taken 3 times and the mean measurement was recorded. The control group mice [FIG. 6C] showed elevated IOP. Another group of mice was treated with intracameral injection of adeno-associated virus (AAV) containing the EcMscL-mCherry gene into the anterior chamber of the eye (AAV2/8-pMGP-EcMscL-mCherry (6.75×10¹⁰ gc/eye)). The needle was inserted through the cornea, anterior to the iridocorneal angle, and 3 μl of virus was pumped into the eye at a rate of 1 μl/min. The needle was left in place in the eye for 1 min after injection and then slowly retracted. IOP was taken before treatment and weekly after that. The IOP was found to decrease [FIG. 6D, FIG. 6E] 3 weeks after intracameral injection of virally carried EcMscL targeted to the TM as compared to untreated control mice group [see FIG. 6C). At the end of the study, mice were sacrificed, and their eyes sectioned for immunohistochemical analysis. Immunostaining with anti-mCherry antibody showed that EcMscL-mCherry was successfully expressed in the TM [see FIG. 6A, FIG. 6B]. The results of this study show that the expression of EcMscL in the TM can be correlated with reduction in elevated IOP and demonstrate the utility of mechanosensitive channels in the treatment of glaucoma. Thus, the heterologously expressed mechanosensitive channels, such as bacterial mechanosensitive channels (MscL) or their generated site-directed mutants, used as osmoregulators, are activated by internal or external mechanical stimuli, and alternative outflow/inflow pathway for fluid in the treatment and prevention of edemas such as passive subretinal, cystoid macular, lymph-, peripheral, pulmonary, and pedal edemas as well as osteoarthiritis related swollen knees and joints.

Example 5—The experiment outlined below demonstrates that EcMscL(I113L/I70E)-mCherry (See SEQ ID NO. 5) can be delivered to the spatially targeted tissue area of an organ in-vivo by non-viral methods. The laser assisted gene delivery method used is called nano-enhanced optical delivery (NOD) wherein low power continuous wave laser beam is enhanced locally due to surface plasmon resonance near the tissue by the gold nanoparticles to create transient perforation of cell membrane by localized temperature rise at hotspots. Though any tissue can be targeted, the retina was used as an example. First, the mouse eye was injected intravitreally with 1 μl of gold nanorods (GNRs). After ˜30-45 min, the eye was injected intravitreally again with 1 μl of plasmid DNA containing the EcMscL(I113L/I70E) gene. Using an OCT (optical coherence tomography) guided continuous wave laser beam, the retina of the eye was irradiated in a spatially controlled manner. Two weeks after the irradiation, mice were sacrificed, their eyes fixed and retina flat-mounts made for immuno-histochemical analysis. Immunostaining with anti-mCherry antibody showed EcMscL(I113L/I70E)-mCherry expression in the treated eye [FIG. 7] and no expression in control eyes.

Example 6—The experiment outlined below demonstrates that EcMscL-mCherry gene (see SEQ ID NO. 1) can be expressed in the mouse brain following intravenous injection of the adeno-associated virus (AAV) carried EcMscL-mCherry gene. Virus was administered through the lateral tail vein. First, a lamp was used to heat the tail and visualize the vein. Then, ˜100 μl of virus, a total of ˜1×10¹¹ vg, was injected into the lateral tail vein (AAV2/8-pMGP-EcMscL-mCherry). Four weeks after the injection, mice were sacrificed, their brains fixed and sectioned for immunohistochemical analysis. Immunostaining with an anti-mCherry antibody showed EcMscL expression at the edge of the cerebellum [FIG. 8A, FIG. 8B] and in the Pons and medulla regions [FIG. 8C, FIG. 8D]. These results demonstrate that virus injected intravenously can cross the blood brain barrier and lead to transduction of MscL expression in the brain.

Example 7—The experiments outlined below demonstrate that Mycobacterium tuberculosis MscL (MtMscL) can be expressed and function in mammalian cells and can be used for molecular delivery and osmotic pressure control. The MscL gene from the H37Rv Mycobacterium tuberculosis strain, which encodes for a 151 amino acid monomer, was codon optimized for mammalian cell expression, N-terminally fused to a signal peptide and C-terminally fused to mCherry (See SEQ ID NO. 2). The signal peptide was used to target the protein to the plasma membrane and the fluorescent reporter was chosen to act as a visual marker of gene expression and membrane localization. HEK293 cells were transfected with the construct and were cultured in 1 mL of standard DMEM growth media in 35 mm petri dishes. A lipofection agent was used to deliver the plasmid DNA to the cells. Fluorescent confocal microscopy of live cells was used to confirm the successful transfection and expression of the fluorescent reporter linked gene. 560-660 nm red emission filter was used to visualize mCherry fluorescence. A population of MtMscL-mCherry transfected HEK293 cells [FIG. 9A] were fluorescent, thereby indicating that MtMscL can be expressed in mammalian hosts. Additionally, the florescence was predominantly seen in the plasma membrane of the cells, thereby indicated successful membrane targeting by the signal peptide.

MtMscL channel function in mammalian cells was also probed by recording electrophysiological measurements from whole cell patches of HEK293 cells expressing MtMscL-mCherry. The patch-clamp recording setup includes an inverted Nikon fluorescence microscope (TS 100) platform using an amplifier system (Axon Multiclamp 700B, Molecular Devices). Micropipettes were pulled using a two-stage pipette puller (Narshinghe) to attain resistance of 3 to 5 MΩ when filled with a solution containing (in mM) 130 K-Gluoconate, 7 KCl, 2 NaCl, 1 MgCl2, EGTA, 10 HEPES, 2 ATP-Mg, 0.3 GTP-Tris and 20 sucrose. The micropipette-electrode was mounted on a micromanipulator. The extracellular solution contained (in mM): 150 NaCl, 10 Glucose, 5 KCl, 2 CaCl2), 1 MgCl2 was buffered with 10 mM HEPES (pH 7.3). Currents were measured while holding cells in voltage clamp at −70 mV. The electrophysiological signals from the amplifier were digitized using Digidata 1440 (Molecular devices), interfaced with patch-clamp software (Clampex, Molecular Devices). pClamp 10 software was used for data analysis. Channel activity was induced by subjecting the patched cell to a hypo-osmotic shock by addition of 20-40% v/v water. A stable gigaohm seal was achieved and the current traces show channel activity only after the hypo-osmotic shock [FIG. 9B], demonstrating that the MtMscL channel is functional in mammalian cells.

HEK293 cells expressing MtMscL-mCherry were subjected to a hypo-osmotic shock in the presence of the membrane impermeable dye, Alexa Fluor™ 488 carboxylic acid succinimidyl ester, by the addition of 20-40% water v/v. The cells were then monitored by confocal fluorescence microscopy for 10-25 minutes in red (543 nm excitation and 560-660 nm emission for mCherry) and green (488 nm excitation and detection through a 505-525 nm emission filter for the membrane impermeable dye) channel [FIG. 9C, overlay of green and red channels]. Analysis of fluorescence images show that after hypo-osmotic shock, only red (mCherry) fluorescent MtMscL expressing cells uptake the green impermeable dye [FIG. 9D]. Non-fluorescent cells stay swollen and dark after the osmotic down shock indicating there was no MtMscL activity (i.e., no transport of extra-cellular impermeable dye). Therefore, we can deduce that red fluorescence is correlated with MtMscL activity and that transfection with the MtMscL-mCherry construct led to the expression of a functional membrane-integrated MtMscL-mCherry channel protein capable of osmoregulation and molecular delivery.

Example 8—The experiments outlined below demonstrate that a variant of E. coli MscL (EcMscL), EcMscL(I113L/I70E) can be expressed and function in mammalian cells, can be used for molecular delivery and osmotic pressure control and has different gating kinetics from the wild-type channel. The MscL gene from the BL21(DE3) E. coli strain, which encodes for a 136 amino acid monomer, was codon optimized for mammalian cell expression, N-terminally fused to a signal peptide, C-terminally fused to mCherry and the I113 and I70 residues mutated (See SEQ ID NO. 5). The signal peptide was used to target the protein to the plasma membrane, the fluorescent reporter was chosen to act as a visual marker of gene expression and membrane localization and the mutations were made to modify channel kinetics. HEK293 cells were transfected with the construct and were cultured in 1 mL of standard DMEM growth media in 35 mm petri dishes. A lipofection agent was used to deliver the plasmid DNA to the cells. Fluorescent confocal microscopy of live cells was used to confirm the successful transfection and expression of the fluorescent reporter linked gene. A 560-660 nm red emission filter was used to visualize mCherry fluorescence. A population of HEK293 cells [FIG. 10A] were fluorescent, thereby indicating that EcMscL(I113L/I70E) can be expressed in mammalian hosts.

EcMscL(I113L/I70E) (See SEQ ID NO. 5) channel function in mammalian cells was probed by recording electrophysiological measurements from whole cell patches of HEK293 cells expressing EcMscL(I113L/I70E)-mCherry. The patch-clamp recording setup includes an inverted Nikon fluorescence microscope (TS 100) platform using an amplifier system (Axon Multiclamp 700B, Molecular Devices). Micropipettes were pulled using a two-stage pipette puller (Narshinghe) to attain resistance of 3 to 5 MΩ when filled with a solution containing (in mM) 130 K-Gluoconate, 7 KCl, 2 NaCl, 1 MgCl2, 0.4 EGTA, 10 HEPES, 2 ATP-Mg, 0.3 GTP-Tris and 20 sucrose. The micropipette-electrode was mounted on a micromanipulator. The extracellular solution contained (in mM): 150 NaCl, 10 Glucose, 5 KCl, 2 CaCl2), 1 MgCl2 was buffered with 10 mM HEPES (pH 7.3). Currents were measured while holding cells in voltage clamp at −70 mV. The electrophysiological signals from the amplifier were digitized using Digidata 1440 (Molecular devices), interfaced with patch-clamp software (Clampex, Molecular Devices). pClamp 10 software was used for data analysis. Channel activity was induced by subjecting the patched cell to a hypo-osmotic shock by addition of 20-40% v/v water. A stable gigaohm seal was achieved and the current traces show channel activity only after the hypo-osmotic shock [FIG. 10B], demonstrating that the EcMscL(I113L/I70E) channel is functional in mammalian cells.

HEK293 cells expressing EcMscL(I113L/I70E)-mCherry (monitored via a red emission filter: 560-660 nm) were subjected to a hypo-osmotic shock in the presence of the membrane impermeable dye, Alexa Fluor™ 488 carboxylic acid succinimidyl ester, by the addition of 20-40% water v/v. The cells were then monitored by confocal fluorescence microscopy for 10-minutes at 488 nm excitation and detected through a green emission filter (505-525 nm). Analysis of fluorescence images show that after hypo-osmotic shock, only red fluorescent EcMscL(I113L/I70E) expressing cells uptake the green dye [FIG. 10C]. Non-fluorescent cells stay swollen and dark after the osmotic down shock indicating there was no EcMscL(I113L/I70E) activity (i.e. no transport of extra-cellular impermeable dye). Therefore, we can deduce that red fluorescence is correlated with EcMscL(I113L/I70E) activity and that transfection with the EcMscL(I113L/I70E)-mCherry construct led to the expression of a functional membrane-integrated EcMscL(I113L/I70E)-mCherry channel protein capable of osmoregulation and molecular delivery.

Example 9—The experiments outlined below demonstrate that Vibrio cholerae MscL (VcMscL) can be expressed and function in mammalian cells and can be used for molecular delivery and osmotic pressure control. The MscL gene from the O395 V. cholerae strain, which encodes for a 136 amino acid monomer, was codon optimized for mammalian cell expression, N-terminally fused to a signal peptide and C-terminally fused to mCherry (See SEQ ID NO. 2). The signal peptide was used to target the protein to the plasma membrane and the fluorescent reporter was chosen to act as a visual marker of gene expression and membrane localization. HEK293 cells were transfected with the construct and were cultured in 1 mL of standard DMEM growth media in 35 mm petri dishes. A lipofection agent was used to deliver the plasmid DNA to the cells. Fluorescent confocal microscopy of live cells was used to confirm the successful transfection and expression of the fluorescent reporter linked gene. The samples were excited at 543 nm and a 560-660 nm red emission filter was used to visualize mCherry fluorescence. A population of HEK293 cells [FIG. 11A] were fluorescent, thereby indicating that VcMscL can be expressed in mammalian hosts. HEK293 cells expressing VcMscL-mCherry (monitored via a red emission filter: 560-660 nm) were subjected to a hypo-osmotic shock in the presence of Alexa Fluor™ 488 carboxylic acid succinimidyl ester by the addition of 20-40% water v/v. The cells were then monitored by confocal fluorescence microscopy for 10-25 minutes at 488 nm excitation and detected through a green emission filter (505-525 nm) [FIG. 11A]. Analysis of fluorescence images show that after hypo-osmotic shock, only red fluorescent VcMscL expressing cells uptake the green dye [FIG. 116]. Non-fluorescent cells stay swollen and dark after the osmotic down shock indicating there was no VcMscL activity (i.e., no transport of extra-cellular impermeable dye). Therefore, we can deduce that red fluorescence is correlated with VcMscL activity and that transfection with the VcMscL-mCherry construct led to the expression of a functional membrane-integrated VcMscL-mCherry channel protein capable of osmoregulation and molecular delivery.

Example 10—The experiment outlined below demonstrates that EcMscL-mCherry gene (see SEQ ID NO. 1) can be expressed in the mouse blood vessels. In control mice, immunostaining with an anti-mCherry antibody showed no expression of mCherry around blood vessels [FIG. 12A]. EcMscL expression in the boundary of the blood vessels of mice transfected with EcMscL-mCherry (I113L/I70E, SEQ ID No. 5) genes [FIG. 12B]. Such bacterial mechanosensitive channels expressing in blood vessels allows activation by internal or external mechanical stimuli for allowing delivery of therapeutic drugs to the target organ of interest as well as for clearance of toxins. Thus, this method allows removal of Beta-Amyloid, Tau, Alpha-synuclein and PolyQ from central nervous system to the blood stream via the blood brain barrier (BBB), the blood-retinal barrier and the blood-spinal cord barrier in neurodegenerative diseases including but not limited to Alzheimer's, Parkinson's, and Huntington's disease.

Example 11—The experiment outlined below demonstrates that MscL when expressed in mammalian cells, generate burst of electrical activities in hypotonic environment. HEK293 cells were cultured in standard DMEM growth media on multi-electrode array (MEA) pertri dishes and a lipofection agent was used to transfect the cells with EcMscL-mCherry gene (I113L/I70E, SEQ ID No. 5) plasmid DNA. Extracellular potential of EcMscL expressing HEK293 cells grown on a multi-electrode array (MEA) petri dish was measured in absence and presence of hypotonic shock (addition of 20% v/v water). Burst of spikes can be seen in the signal during 13-14.5 sec [FIG. 13], demonstrating robust EcMscL channel activities. This result in combination with the result shown in FIG. 4G shows that MscL or their generated site-directed mutants, when expressed in cells act as osmoregulators and generates electrical activities in response to osmotic shocks. Thus, the Hematopoietic stem cells expressing MscL will generate red blood cells which act as osmoregulators and resistant to osmotic shock, in the treatment and prevention of hyper- as well as hypo-tension related diseases.

Example 12—The experiment outlined below demonstrates that E. coli MscL (EcMscL) channel activity can be induced in HEK cells (model for excitatory cells such as neuron and cardiac cells) by ultrasound stimulation. HEK293 cells were cultured in standard DMEM growth media on multi-electrode array (MEA) pertri dishes and a lipofection agent was used to transfect the cells with plasmid DNA. The MEA plate was placed on an ultrasound device and was set up to record electrical signals from all electrodes simultaneously. Therefore, any channel activity in cells attached to an electrode would lead to a spike in the electrical signal from that electrode. Ultrasound stimulation (Pulse width: 250 ms, Repetition rate: 2 Hz, Frequency: 1.1 MHz) was used to stimulate the cells on the MEA plate. No electrical activities were observed in control cells (not transfected with EcMscL) stimulated by ultrasound or in EcMscL-transfected cells without ultrasound stimulation [FIG. 14A]. The electrical recordings show a spike in signal on some electrodes after ultrasound stimulation [FIG. 14B]. The results of the experiment confirm that MscL channel activity can be stimulated by ultrasound. Thus, heterologously expressed mechanosensitive channels, such as bacterial mechanosensitive channels or their generated site-directed mutants, in neurons are activated by internal or external mechanical stimuli, for modulating activities in dormant neurons in order to recover from coma, and persistent vegetative state. Thus, heterologously expressed mechanosensitive channels, or their generated site-directed mutants, in neurons can be activated by internal or external mechanical stimuli, for modulating neural activities in order to repair injury such as concussion, enhance neural regeneration, accelerated learning and memory processing. Efficiency of such external stimulation process can be enhanced with or without conjugation of nanoprobes, wherein the device providing external mechanical force or magnetic field can be controlled to tune the duration, frequency and strength of stimulation. This will allow control on molecular delivery, stimulation or cellular death leading to the desired therapeutic outcome.

Example 13—The experiment described below was conducted to test if the mechanosensitive channel (MscL) can be expressed in corneal epithelial cells, so that those cells' activities can be modulated for alleviation of dry eye disease (DED). The mice were treated with intracameral injection of adeno-associated virus containing the EcMscL-mCherry gene (AAV2/8-EcMscL-mCherry, 6.75×10¹⁰ vg/eye) into the anterior chamber of the eye. The needle was inserted through the cornea, anterior to the iridocorneal angle, and 3 μl of virus was pumped into the eye at a rate of 1 μl/min. The needle was left in place in the eye for 1 min after injection and then slowly retracted. FIG. 15 shows the confocal Image of a section of the mouse cornea transfected with EcMscL-mCherry. Expression of reporter-mCherry exhibited red fluorescence, detected by 543 nm excitation and 560-660 nm emission. Using this viral transduction method, meibomian glands endothelial cells can also be made to express MscL, which can be stimulated mechanically (as demonstrated in model HEK cells in FIG. 14B) that would lead to enhance in secretion of aqueous phase of the tear film and thus, alleviation of dry eye disease (DED).

Example 14—The example described below demonstrates that the MscL-mCherry gene can be expressed in the Heart and other vital organs such as the Kidney in order to allow modulation of cellular function and associated therapeutic outcome. The adeno-associated virus (AAV) carried EcMscL-mCherry gene was administered through the lateral tail vein. First, a lamp was used to heat the tail and visualize the vein. Then, ˜100 μl of virus, a total of ˜1×10¹¹ vg, was injected into the lateral tail vein. Four weeks after the injection, mice were sacrificed, their Hearts and Kidneys fixed and sectioned for immunohistochemical analysis. Immunostaining with an anti-mCherry antibody showed EcMscL expression in the Heart [FIG. 16A]. FIG. 16B shows confocal Image of the mouse Kidney transfected with EcMscL-mCherry via AAV based gene transduction. Expression of reporter-mCherry exhibited by characteristic red fluorescence in 560-660 nm emission band. Thus, the cardiac or kidney cells expressing mechanosensitive channels (e.g., MscL) or their generated site-directed mutants can be activated by internal or external mechanical stimuli, and used as osmoregulators or diuretics in the renal system for the treatment and prevention of kidney stones and chronic kidney disease.

Example 15—The example described below demonstrates that MscL-mCherry gene can be expressed in the Liver to allow modulation of cellular function and associated therapeutic outcome. To achieve transduction in Liver, the adeno-associated virus (AAV) carried EcMscL-mCherry gene was administered through the lateral tail vein. First, a lamp was used to heat the tail and visualize the vein. Then, ˜100 μl of virus, a total of ˜1×10¹¹ vg, was injected into the lateral tail vein. Four weeks after the injection, mice were sacrificed, their Livers fixed and sectioned for immunohistochemical analysis. Immunostaining with an anti-mCherry antibody showed EcMscL expression in the Liver [FIG. 16C] transfected with EcMscL-mCherry via AAV based gene transduction. Expression of reporter-mCherry exhibited by characteristic red fluorescence. The heterologously expressed mechanosensitive channels, such as bacterial mechanosensitive channels (MscL), or their generated site-directed mutants expressed in liver, gallbladder or bile duct cells of the hepatic system can thus be used as bile regulators, activated by internal or external mechanical stimuli for the treatment and prevention of gallstones.

Example 16—The example described below demonstrates that MscL-mCherry gene can be expressed in the Lung to allow modulation of cellular function and associated therapeutic outcome. To achieve transduction in the Lung, the adeno-associated virus (AAV) carried EcMscL-mCherry gene was administered through the lateral tail vein. First, a lamp was used to heat the tail and visualize the vein. Then, ˜100 μl of virus, a total of ˜1×10¹¹ vg, was injected into the lateral tail vein. Four weeks after the injection, mice were sacrificed, their Lungs fixed and sectioned for immunohistochemical analysis. Immunostaining with an anti-mCherry antibody showed EcMscL expression in the Lung [FIG. 16D] transfected with EcMscL-mCherry via AAV based gene transduction. Expression of reporter-mCherry exhibited by red fluorescence. The mechanosensitive channels, such as bacterial mechanosensitive channels (MscL), or their generated site-directed mutants, heterologously expressed in Pneumocytes, can be used as ports for exchange of oxygen and Carbon dioxide from alveolus to the blood capillaries and vice a versa. The activities of these channels/ports on Pneumatocytes can be further modulated by internal or external mechanical stimuli for treatment of lung diseases including but not limited to Black Lung Disease (Coal workers' pneumoconiosis).

Example 17—The example described below demonstrates the electrical activity of mammalian cells expressing EcMscL^{m}-mCherry gene on a membrane to varied mechanical pressures. To evaluate the functioning of EcMscL^{m} in mammalian (e.g., Human Embryonic Kidney: HEK) cells, EcMscL^{m}-transfected cells and non-transfected (−ve control) cells were subjected to pressure clamp electrophysiology. Automated pressure-clamp electrophysiology was used to measure the effect of different mechanical pressures on EcMscL^{m} expressing mammalian cells. FIG. 17A. shows a fluorescence (mCherry-reporter) image of HEK cells expressing EcMscL^{m}. The EcMscL^{m} transfected cells were transferred to patch clamp chamber in which negative pressure was created by use of a suction pump to exert a definite pressure on the cells. Representative inward current profiles of EcMscL^{m} expressing cell subjected to −15 mmHg and −30 mmHg are shown in FIG. 176. The cells not transfected (−ve control) with EcMscL^{m} did not generate any current response even when subjected to −30 mmHg (inset, [FIG. 17B]). FIG. 17C shows the quantitative comparison of inward peak current for 2 different hold-pressures as compared to no pressure control in EcMscL^{m}-transfected cells, and −ve control (non-transfected) cells. Statistically significant different in pressure-sensitive changes in current was observed in EcMscL^{m} transfected cells. EcMscL^{m}-channel expressing cell was found to respond to sharp (˜50 ms) pressure changes [FIG. 17D].

Example 18—The example described below demonstrates EcMscL^{m} gene expression in cells does not compromise cell survival. To evaluate survivability of EcMscL^{m} expressing cells in-vivo, an intracameral injection of AAV2/8-pMGP-EcMscL^{m}-mCherry (vEcMscL^{m}: 6.75×10¹⁰ vg/eye) was carried out in mice. Intracameral AAV-EcMscL^{m}-mCherry (vEcMscL^{m}) injection in mice led to EcMscL^{m}-mCherry (intrinsic) expression in iridocorneal junction [FIG. 18A]. Representative fluorescence images of a flat mount anterior segment showing EcMscL^{m}-mCherry (intrinsic) expression in iridocorneal junction in DEX-injected IOP elevated mice 4 weeks post intracameral AAV-EcMscL^{m}-mCherry (vEcMscL^{m}) injection. Representative fluorescence image of a flat mount anterior segment [FIG. 18B] shows no red fluorescence in iridocorneal junction in DEX-only control mice. FIG. 18C shows the bar plot of intensity of mCherry-reporter signal in the trabecular meshwork for AAV-EcMscL^{m}-mCherry-treated and control eyes. Representative fluorescence image of anterior chamber flat mount, immunostained with mCherry (red, reporter for EcMscL^{m}), is shown in FIG. 18D for AAV-EcMscL^{m}-mCherry-treated mouse. To assess cell survival in EcMscL^{m} expressing mice, the anterior chamber was also immunostained with an apoptotic marker. FIG. 18E shows the representative fluorescence image of an anterior chamber flat mount, immunostained with Caspase-3 (green), for AAV-EcMscL^{m}-mCherry (vEcMscL^{m}) treated DEX-mouse. No detectable apoptotic cells in vEcMscL^{m} treated mice was detected. Representative fluorescence images of an anterior chamber flat mount immunostained with Caspase-3 (green), for control wild type −ve control (PBS injected) mouse, is shown in FIG. 18F. FIG. 18G and FIG. 18H respectively show Myocilin (TM-marker) and mCherry-reporter expression in the Iridocorneal regions in the axial section of eye transduced by vEcMscL^{m}. Western Blot confirmed EcMscL^{m} (estimated Molecular weight=˜42 kDa) expression in mice anterior chamber lysate [FIG. 18I].

Example 19—The example described below demonstrates the mechanism via which the expressed EcMscL^{m} gene on cell membrane leads to modulation of pressure in an organ as evidenced in modulation of fluid transport measured by aqueous humor (AH) outflow facility. To measure aqueous outflow facility, the mice were anesthetized. The anterior chamber of the mouse eye is cannulated by using a 32-gauge steel needle [FIG. 19A], which was connected to a flow-through pressure transducer for the continuous determination of pressure within the system. In the anesthetized animals, pressure was monitored continuously as the Flow rate was set at different rates. The pump was stopped, and the circuit opened to the manometer, which was then rapidly lowered to re-establish baseline pressure. FIG. 19B shows the measured outflow in DEX-treated control vs. intracameral AAV-EcMscL^{m}-mCherry (vEcMscL^{m})-injected DEX-mice. As can been seen, the mice expressing EcMscL^{m} exhibited increase in AH outflow facility over the control mice.

Example 20—The example described below demonstrates that EcMsCl^{M} gene expression provides neuroprotection against abnormal pressure condition. Mice eyes were treated with vehicle or vEcMscL^{m} (following 4 weeks of intracameral injection in post-DEX C57BL/6J mice) in Dexamethasone treated eyes. To evaluate if intracameral injection of vEcMscL^{m} protects the RGC by lowering IOP, visually evoked potential (VEP) measurements were carried out in DEX-control and DEX+vEcMscL^{m} groups. VEP of DEX-mice and DEX-mice injected with vEcMscL^{m} at light intensity of 7×10¹³ photons cm⁻² s⁻¹, 5 weeks after vehicle injection are shown in FIG. 20A & FIG. 20B respectively. As shown in these figures, the VEP amplitude (˜15 mV) is much lower than typical VEP measured in wild type control mice (˜50 mV). Quantitative comparison shows significantly higher VEP-amplitude in vEcMsCl^{m} injected group as compared to DEX-only mice, 5 weeks after injection (FIG. 20C). The mean difference of VEP amplitude between non-transfected and vEcMscL^{m} injected DEX-mice is shown as the Gardner-Altman estimation plot [FIG. 20D]. The curve indicates the resampled distribution of the mean difference, given the observed data. The mean difference was plotted on floating axes on the right as a bootstrap sampling distribution. The mean difference is depicted as a dot; the 95% confidence interval is indicated by the ends of the vertical error bar. FIG. 20E shows the RBPMS (RGC marker) immunostained fluorescence images in peripheral and central retina of DEX-treated mice eyes without and with vEcMscL^{m} treatment (following 4 weeks of intracameral injection) in post-DEX C57BL/6J mice. RGCs labeled with RGC marker RBPMS, in red, counted to determine the surviving RGCs. Quantitative comparison of RGC counts per mm² of the retina of DEX-control and vEcMscL^{m} transduced DEX-mice [FIG. 20F] shows that vEcMscL^{m} provides neuroprotection against abnormal pressure condition.

Example 21—The example described below demonstrates that the expression of EcMscL^{m} gene in-vivo does not elicit immune response. To evaluate if vEcMscL^{m} injection leads to local immune response, the anterior chamber of vEcMscL^{m}-injected mice was immunostained with Iba1 (marker for microglia) and IFNg (inflammatory cytokine). Immunostained (Iba1) image FIG. 21A of AAV-EcMscL^{m}-mCherry (vEcMscL^{m})-injected mice shows no immune cell response in the injected organ. FIG. 21B. shows minimal (basal) level of fluorescence in immunostained (IFNg) image of vEcMscL^{m}-injected mice implying absence of this inflammatory cytokine in the injected organ. For comparison, Iba1 and IFNg immunostained images of anterior chamber of control wild type mice are shown in FIG. 21C and FIG. 21D, respectively. FIG. 21E shows the quantitative comparison of IL-6 (pro-inflammatory marker) in plasma between baseline and after 1 and 10 weeks of vEcMscL^{m} transduction. No statistically significant difference implies no systemic immune response in vEcMscL^{m} injected mice.

Example 22—The example described below demonstrates the method by which EcMscL^{m}-mCherry expression in endothelial cells can control the hypertension. Here, as an example, the effect of EcMscL^{m}-mCherry expression in endothelial cells of the glomerulus or the excretion apparatus in the kidney on reducing hypertension is examined. Hypertensive BPH/2J mice were anesthetized with isoflurane and warmed for 5-10 minutes to dilate the veins. The needle (small gauge, 27-30) was inserted, bevel up, into the vein towards the direction of the head. Approximately 0.25 ml of scAAV-EcMscL^{m}-mCherry (Sequence ID No: 5) was injected into the tail vein of each mouse. The needle was removed, and the mice were placed in a recovery cage. 2 weeks later, the mice were injected with Ketamine-xylazine-acepromazine (KXA) and placed on a warming blanket. The tail temperature was measured to ensure that it was at least 32° C. and the occlusion cuff and volume sensing cuffs (in that order) were placed on the mouse until the first sign of resistance. The Kent scientific system was used to measure blood pressure for at least 15 cycles (5 acclimatization, 10 measurements). After measurements, the mouse was injected intraperitoneally with 0.5 ml of normal saline. FIG. 22A shows the tail-cuff blood pressure monitoring method by use of Volume Pressure Recording (VPR). The quantitative comparison of systolic and diastolic blood pressure in EcMscL^{m}-mCherry (Double mutant, I113L/I70E) treated and untreated mice model of elevated blood pressure (HTN model) is shown in FIG. 22B. WT mouse data having normal blood pressure is included for comparison. FIG. 22C shows the EcMsCl^{M}-mCherry (red) immunostained image of kidney tissue of vEcMscL^{m}-mCherry injected mice. As shown, robust expression of EcMscL^{m}-mCherry in membrane was observed in contrast to that of kidney tissue of non-injected HTN mouse [FIG. 22D]. The results demonstrated the expression of EcMscL^{m}-mCherry in membrane of endothelial cells leads to regulation of the blood pressure.

Example 23—The example described below demonstrates that heterologous expression of mechanosensitive channel in selected areas of nervous system in conjunction with external (ultrasound) stimulation can lead to efficient pain modulation. The wild type mice were anesthetized with 2-3.5% isoflurane and the fur over the area of interest was removed chemically. A midline incision is made, and the skin removed in the anterior cingulate cortex (ACC) area. A burr hole is made over the ACC (0.7 mm anterior to bregma, 0.4 mm lateral from the midline, and at a depth of 1.8 mm from the skull surface) and 3 microliters of 10¹⁰ vg/mL vEcMscL^{m}-mCherry virus was injected into the ACC. The mice were maintained for 2 weeks to allow proper expression. An ultrasonic probe outside the skull was used to stimulate the transduced ACC region [FIG. 23A]. Mice paws were then given a 10 microliter injection of 1% Formalin (to induce inflammatory pain) and placed in a holding chamber. At 5-minute intervals, for 1 minute each, mice were observed for paw lifting and paw licking behavior. The total time in seconds, wherein this behavior was observed, for each minute of observation was recorded. This was continued for 45 minutes. Pain scoring was performed according to the following formula: (2×Paw licking time+1×Paw licking time)/60. FIG. 23B shows the acute pain responses are reduced with ultrasound stimulation of the vEcMscL^{m} treated ACC region.

Example 24—This example describes the effect of EcMscL^{m}-mCherry expression on regulation of fibroproliferation and related abnormal physiological parameter(s). TGFβ is known to cause fibroproliferation, leading to intraocular pressure (IOP) rise when injected in eye. An adenovirus 5 (Ad5) viral vector was used to over-express a bioactivated form of human TGFβ2 in the trabecular meshworks TM of mouse eyes. Briefly, mice were first anesthetized with 2.5% isoflurane+100% oxygen. Anesthetized mice were then given topical 0.5% proparacaine to numb the eye. The Ad5-CMV-TGFβ2^{c226s/c228s} virus or Ad5 null virus containing no transgene (EcMscL^{m}) was intravitreally injected using 1.5 μl of 1×10¹² plaque forming units/ml with a 10 μl Hamilton syringe and a 33 g beveled needle (World Precision Instruments). 5 Wildtype C57BL/6J mice per group and a total of 4 groups were utilized. The first group included TGFb/Control AAV2-mCherry, the second group included TGFβ-2/scEcMscL^{m}-mcherry Virus, the third group included Ad5/Control AAV2-mCherry, and the fourth group included AD5/scEcMscL^{m}-mCherry Virus. All groups had 2 baselines of IOP measured. The first 2 groups (1, 2) were intravitreally injected with TGFβ2 first, and the last 2 group (3, 4) were injected with Adeno 5 Virus. After 2 weeks of injection with either the TGFβ2 or the Ad5-control, intracameral injection of the scEcMscL^{m}-mCherry Virus (2.96×10¹⁰ vg/ml) or the control AAV2-mCherry (8.6×10¹⁰ vg/ml) was performed. The IOP was measured at regular time points. FIG. 24 shows the comparison of IOP(mmHg) measurement for TGFb (fibrosis upregulator) and Control Adeno 5 Virus models after EcMscL^{m}-mCherry expression in IOP elevated mice. As seen from the results [FIG. 24], it is shown that that the control group with TGFb injection led to increased IOP whereas the scEcMscL^{m}-mCherry injection led to decrease in IOP. This result shows the effect of EcMscL^{m}-mCherry in suppression of fibrogenic activity of TGFb.

Example 25—The example describes the Ultrasound Stimulation based neuromodulation of EcMscL^{m}-mCherry expressing cells in the brain. EcMscL^{m}-mCherry (˜2E10 vg) virus was injected to wildtype mice to transduce the cortical neurons resulting in expression of EcMscL^{m}-mCherry in the cell membrane. A single channel transduced was mounted on top of the EcMscL^{m}-transduced cortex and the gap between the transducer and cortical surface was filled using Saline/Genteal to allow propagation of ultrasound pulse. A train of 25 MHz, 20 mV ultrasound pulse was applied and cortical activity was recorded using a multi electrode array (MEA). The recording was performed using Plexon MEA System. Offline sorter was used on the wide band (WB) waveform traces to detect and quantify the spikes. FIG. 25A shows the in-vivo MEA recording of ultrasound stimulation (25 MHz, 20 mV, 5 ms ON) evoked spiking in neurons with and without EcMscL^{m} sensitization. The quantitative comparison of spiking rate generated in EcMscL^{m}-transduced neurons in-vivo with different intensities of ultrasound stimulation is shown in FIG. 25B. Also shown is the ultrasound (20 mV) stimulated value from wildtype control with no EcMscL^{m} transduction. The significantly enhanced spiking activities by low power ultrasound stimulation of targeted EcMscL^{m}-expressing neurons in the brain demonstrates the potential of this method in efficient neuromodulation.

Example 26—The example describes the neuromodulation of retinal cells in the eye by expression of mechanosensitive channel combined with Ultrasound Stimulation (US). EcMscL^{m} (1.5 microliter/eye) virus was injected intravitreally to rd1 mice eye (lacking photoreceptors) to transduce the retinal neurons with EcMscL^{m}. A single channel US-transducer was placed close to the cornea. The gap between the transducer and corneal surface was filled using Genteal to allow propagation of ultrasound pulse. A train of 3.1 MHz 20 mV ultrasound pulse (10 ms) was applied on the retinal neurons and retinal activity was recorded using electrophysiology system (NS-NEEL, Nanoscope Instruments Inc). The signal was averaged (15 recordings) and filtered for the analysis and quantification of US-evoked electrical signal. Transduction of retinal ganglion cells (RGCs) with EcMscL^{m} is shown in FIG. 26A. Reporter (mCherry) expression (red) overlaid with Thy1-RGC marker (green) is shown. In-vivo electrical response upon 3.1 MHz ultrasound (20 mV) stimulation of eye of rd1 mice at cornea with and without EcMscL^{m} sensitization of retina is shown in FIG. 26B. Quantitative comparison of amplitudes of US-evoked electrical signal showed significantly larger values for EcMscL^{m}-transduced retina vs. −ve control (rd1 mice without EcMscL^{m} transduction).

Example 27—This example demonstrates the application of the heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, to be used as a tension activated pressure release valve in cells in diseases characterized by elevated pressure or impaired permeability of tissues. Some of the examples wherein the expressed mechanosensitive channels would therapeutically benefit includes but not limited to Glaucoma (Elevated Intraocular pressure), Stroke/Seizure (Intracranial pressure rise), Hypertensive encephalopathy (Elevated Hydrostatic force), Heart Failure (Interstitial tissue pressure), Kidney disease (Interstitial Fluid accumulation), Erectile dysfunction (Blood flow abnormality), Lymphedema (Inadequate drainage of lymph fluid), Menopause (Stoppage of menstrual flow), and Hypertensive cardiomyopathy (Diastolic/systolic dysfunction).

Table-01: Amino acid sequence of a synthetic mechanosensitive channel derived from Escherichia coli-MscL with signaling peptides for mammalian plasma membrane targeting.

(SEQ ID No: 1)
Met Leu Pro GIn Gln Val Gly Phe Val Cys Ala Val
Leu Ala Leu Val Cys Cys Ala Ser Gly Met Ser lle
lle Lys Glu Phe Arg Glu Phe Ala Met Arg Gly Asn
Val Val Asp Leu Ala Val Gly Val lle lle Gly Ala
Ala Phe Gly Lys lle Val Ser Ser Leu Val Ala Asp
lle lle Met Pro Pro Leu Gly Leu Leu lle Gly Gly
lle Asp Phe Lys Gln Phe Ala Val Thr Leu Arg Asp
Ala GIn Gly Asp lle Pro Ala Val Val Met His Tyr
Gly Val Phe lle Gln Asn Val Phe Asp Phe Leu lle
Val Ala Phe Ala lle Phe Met Ala lle Lys Leu lle
Asn Lys Leu Asn Arg Lys Lys Glu Glu Pro Ala Ala
Ala Pro Ala Pro Thr Lys Glu Glu Val Leu Leu Thr
Glu lle Arg Asp Leu Leu Lys Glu GIn Asn Asn Arg
Ser

Table-02: Amino acid sequence of a synthetic mechanosensitive channel derived from Mycobacterium Tuberculosis-MscL with signaling peptides for mammalian plasma membrane targeting.

(SEQ ID NO: 2)
Met Leu Pro GIn Gln Val Gly Phe Val Cys Ala Val
Leu Ala Leu Val Cys Cys Ala Ser Gly Met Leu Lys
Gly Phe Lys Glu Phe Leu Ala Arg Gly Asn lle Val
Asp Leu Ala Val Ala Val Val lle Gly Thr Ala Phe
Thr Ala Leu Val Thr Lys Phe Thr Asp Ser lle lle
Thr Pro Leu lle Asn Arg lle Gly Val Asn Ala Gln
Ser Asp Val Gly lle Leu Arg lle Gly lle Gly Gly
Gly Gln Thr lle Asp Leu Asn Val Leu Leu Ser Ala
Ala lle Asn Phe Phe Leu lle Ala Phe Ala Val Tyr
Phe Leu Val Val Leu Pro Tyr Asn Thr Leu Arg Lys
Lys Gly Glu Val Glu GIn Pro Gly Asp Thr Gln Val
Val Leu Leu Thr Glu lle Arg Asp Leu Leu Ala GIn
Thr Asn Gly Asp Ser Pro Gly Arg His Gly Gly Arg
Gly Thr Pro Ser Pro Thr Asp Gly Pro Arg Ala Ser
Thr Glu Ser Gln

Table-03: Amino acid sequence of a synthetic mechanosensitive channel derived from Staphylococcus aureus-MscL with signaling peptides for mammalian plasma membrane targeting.

(SEQ ID NO: 3)
Met Leu Pro GIn Gln Val Gly Phe Val Cys Ala Val
Leu Ala Leu Val Cys Cys Ala Ser Gly Met Leu Lys
Glu Phe Lys Glu Phe Ala Leu Lys Gly Asn Val Leu
Asp Leu Ala lle Ala Val Val Met Gly Ala Ala Phe
Asn Lys lle lle Ser Ser Leu Val Glu Asn lle lle
Met Pro Leu lle Gly Lys lle Phe Gly Ser Val Asp
Phe Ala Lys Glu Trp Ser Phe Trp Gly lle Lys Tyr
Gly Leu Phe lle Gln Ser Val lle Asp Phe lle lle
lle Ala Phe Ala Leu Phe lle Phe Val Lys lle Ala
Asn Thr Leu Met Lys Lys Glu Glu Ala Glu Glu Glu
Ala Val Val Glu Glu Asn Val Val Leu Leu Thr Glu
lle Arg Asp Leu Leu Arg Glu Lys Lys

Table-04: Amino acid sequence of a synthetic mechanosensitive channel derived from Vibrio cholerae-MscL with signaling peptides for mammalian plasma membrane targeting.

(SEQ ID NO: 4)
Met Leu Pro GIn Gln Val Gly Phe Val Cys Ala Val
Leu Ala Leu Val Cys Cys Ala Ser Gly Met Ser Leu
Leu Lys Glu Phe Lys Ala Phe Ala Ser Arg Gly
Asn Val lle Asp Met Ala Val Gly lle lle lle Gly
Ala Ala Phe Gly Lys lle Val Ser Ser Phe Val Ala
Asp lle lle Met Pro Pro lle Gly lle lle Leu Gly
Gly Val Asn Phe Ser Asp Leu Ser Phe Val Leu Leu
Ala Ala GIn Gly Asp Ala Pro Ala Val Val lle Ala
Tyr Gly Lys Phe lle Gln Thr Val Val Asp Phe Thr
lle lle Ala Phe Ala lle Phe Met Gly Leu Lys Ala
lle Asn Ser Leu Lys Arg Lys Glu Glu Glu Ala Pro
Lys Ala Pro Pro Ala Pro Thr Lys Asp Gln Glu Leu
Leu Ser Glu lle Arg Asp Leu Leu Lys Ala GIn Gln
Asp Lys

Table-05: Amino acid sequence of a synthetic mechanosensitive channel derived from Escherichia coli-MscL with signaling peptides for mammalian plasma membrane targeting having mutation (I113L/I70E).

(SEQ ID NO: 5)
Met Leu Pro GIn Gln Val Gly Phe Val Cys Ala Val
Leu Ala Leu Val Cys Cys Ala Ser Gly Met Ser lle
lle Lys Glu Phe Arg Glu Phe Ala Met Arg Gly Asn
Val Val Asp Leu Ala Val Gly Val lle lle Gly Ala
Ala Phe Gly Lys lle Val Ser Ser Leu Val Ala Asp
lle lle Met Pro Pro Leu Gly Leu Leu Glu Gly Gly
lle Asp Phe Lys Gln Phe Ala Val Thr Leu Arg Asp
Ala GIn Gly Asp lle Pro Ala Val Val Met His Tyr
Gly Val Phe lle Gln Asn Val Phe Asp Phe Leu lle
Val Ala Phe Ala Leu Phe Met Ala lle Lys Leu lle
Asn Lys Leu Asn Arg Lys Lys Glu Glu Pro Ala Ala
Ala Pro Ala Pro Thr Lys Glu Glu Val Leu Leu Thr
Glu lle Arg Asp Leu Leu Lys Glu GIn Asn Asn Arg
Ser

Table-06: Amino acid sequence of a synthetic mechanosensitive channel derived from Escherichia coli-MscL with signaling peptides for mammalian plasma membrane targeting having mutation (I113L/K122T).

(SEQ ID NO: 6)
Met Leu Pro GIn Gln Val Gly Phe Val Cys Ala Val
Leu Ala Leu Val Cys Cys Ala Ser Gly Met Ser lle
lle Lys Glu Phe Arg Glu Phe Ala Met Arg Gly Asn
Val Val Asp Leu Ala Val Gly Val lle lle Gly Ala
Ala Phe Gly Lys lle Val Ser Ser Leu Val Ala Asp
lle lle Met Pro Pro Leu Gly Leu Leu lle Gly Gly
lle Asp Phe Lys Gln Phe Ala Val Thr Leu Arg Asp
Ala GIn Gly Asp lle Pro Ala Val Val Met His Tyr
Gly Val Phe lle Gln Asn Val Phe Asp Phe Leu lle
Val Ala Phe Ala Leu Phe Met Ala lle Lys Leu lle
Asn Thr Leu Asn Arg Lys Lys Glu Glu Pro Ala Ala
Ala Pro Ala Pro Thr Lys Glu Glu Val Leu Leu Thr
Glu lle Arg Asp Leu Leu Lys Glu GIn Asn Asn Arg
Ser

The specification and examples herein provide a complete description of the structure and use of illustrative embodiments. Although certain embodiments have been described with a certain degree of particularity, or with reference to one or more individual embodiments, those skilled in the art could make numerous alterations to the disclosed embodiments without departing from the scope of this invention. As such, the various illustrative embodiments of the devices are not intended to be limited to the particular forms disclosed. Rather, they include all modifications and alternatives falling within the scope of the claims, and embodiments other than the one shown may include some or all of the features of the depicted embodiment. For example, components may be omitted or combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the present invention.

Furthermore, the claims are not intended to include, and should not be interpreted to include, means-plus- or step-plus-function limitations, unless such a limitation is explicitly recited in a given claim using the phrase(s) “means for” or “step for,” respectively.

### REFERENCES

The following references, to the extent that they provide exemplary procedural or other details supplementary to those set forth above, are specifically incorporated by reference.

ADDIN Mendeley Bibliography CSL_BIBLIOGRAPHY 1. Glaucoma.org. Glaucoma Facts and Stats.
2. Bermudez, J. Y., Montecchi-Palmer, M., Mao, W. & Clark, A. F. Cross-linked actin networks (CLANs) in glaucoma. Experimental Eye Research (2017). doi:10.1016/j.exer.2017.02.010
3. Radcliffe, N. M. & Kaufman, P. L. Glaucoma Drugs in the Pipeline. Asia-Pacific J. Ophthalmol. (Philadelphia, Pa.) 3-5 (2018). doi:10.22608/AP0.2018298
4. Challa, P. & Arnold, J. J. Rho-kinase inhibitors offer a new approach in the treatment of glaucoma. Expert Opin. Investig. Drugs (2014). doi:10.1517/13543784.2013.840288
5. Dismuke, W. M., Mbadugha, C. C. & Ellis, D. Z. NO-induced regulation of human trabecular meshwork cell volume and aqueous humor outflow facility involve the BKCa ion channel. AJP Cell Physiol. (2008). doi:10.1152/ajpcell.00363.2007
6. Dismuke, W. M., Sharif, N. A. & Ellis, D. Z. Human trabecular meshwork cell volume decrease by NO-independent soluble guanylate cyclase activators YC-1 and BAY-58-2667 involves the BKCa ion channel. Investig. Ophthalmol. Vis. Sci. (2009). doi:10.1167/iovs.08-3127
7. Vasantha Rao, P., Deng, P. F., Kumar, J. & Epstein, D. L. Modulation of aqueous humor outflow facility by the Rho kinase-specific inhibitor Y-27632. Investig. Ophthalmol. Vis. Sci. (2001). doi:10.1016/0022-3697(80)90091-8
8. Zhu, W. et al. Transplantation of iPSC-derived TM cells rescues glaucoma phenotypes in vivo. Proc. Natl. Acad. Sci. U.S.A. 113, E3492-E3500 (2016).
9. Wang, C., Li, L. & Liu, Z. Experimental research on the relationship between the stiffness and the expressions of fibronectin proteins and adaptor proteins of rat trabecular meshwork cells. BMC Ophthalmol. (2017). doi:10.1186/s12886-017-0662-5
10. Ryskamp, D. A. et al. TRPV4 regulates calcium homeostasis, cytoskeletal remodeling, conventional outflow and intraocular pressure in the mammalian eye. Sci. Rep. 6, 30583 (2016).
11. Fernandes, K. A. et al. Using genetic mouse models to gain insight into glaucoma: Past results and future possibilities. Exp. Eye Res. 141, 42-56 (2015).
12. Johnson, T. V & Tomarev, S. I. Rodent models of glaucoma. Brain Res. Bull. (2010). doi:10.1016/j.brainresbull.2009.04.004
13. Zode, G. S. et al. Reduction of ER stress via a chemical chaperone prevents disease phenotypes in a mouse model of primary open angle glaucoma. J. Clin. Invest. (2011). doi:10.1172/JCI58183
14. Kasetti, R. B., Phan, T. N., Millar, J. C. & Zode, G. S. Expression of Mutant Myocilin Induces Abnormal Intracellular Accumulation of Selected Extracellular Matrix Proteins in the Trabecular Meshwork. Invest. Ophthalmol. Vis. Sci. 57, 6058-6069 (2016).
15. Oddsson A, Sulem P, Helgason H, et al. Common and rare variants associated with kidney stones and biochemical traits. Nat Commun. 2015; 6:7975.

What is claimed is:

1. A synthetic polypeptide sequence of a mechanosensitive channel of large conductance 1 (MscL1) protein and its generated site-directed mutant(s) thereof, wherein said MscL1 or mutant(s) thereof comprises at least 75% sequence identity to SEQ ID NO:1 and wherein the MscL1 protein or mutant(s) thereof, when expressed on mammalian cell membrane, senses pressure changes and modulates the intra-cellular pressure, or molecular transport including aqueous fluid and therapeutic molecules.

2. A synthetic polypeptide sequence of MscL2 protein and its generated site-directed mutant(s) thereof, wherein said MscL2 or mutant(s) thereof comprises at least 75% sequence identity to SEQ ID NO: 2 and wherein the MscL2 protein or mutant(s) thereof, when expressed on mammalian cell membrane, senses pressure changes and modulates the intra-cellular pressure, or molecular transport including aqueous fluid and therapeutic molecules.

3. A synthetic polypeptide sequence of MscL3 protein and its generated site-directed mutant(s) thereof, wherein said MscL3 or mutant(s) thereof comprises at least 75% sequence identity to SEQ ID NO: 3 and wherein the MscL3 protein or mutant(s) thereof, when expressed on mammalian cell membrane, senses pressure changes and modulates the intra-cellular pressure, or molecular transport including aqueous fluid and therapeutic molecules.

4. A synthetic polypeptide sequence of MscL4 protein and its generated site-directed mutant(s) thereof, wherein said MscL4 or mutant(s) thereof comprises at least 75% sequence identity to SEQ ID NO: 4 and wherein the MscL4 protein or mutant(s) thereof, when expressed on mammalian cell membrane, senses pressure changes and modulates the intra-cellular pressure, or molecular transport including fluid and therapeutic molecules.

5. The synthetic polypeptide sequence of claim 1 wherein the amino acid sequence comprises of SEQ ID NO:5 or SEQ ID NO:6.

6. The synthetic polypeptide sequence of claim 1, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains are delivered locally, intraocularly, intravenously, intrathecally, intramuscularly or subcapsularly.

7. The synthetic polypeptide sequence of claim 1, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used as a tension activated pressure release valve in cells for reduction of blood pressure.

8. The synthetic polypeptide sequence of claim 7, wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are expressed in endothelial cells of the glomerulus or the excretion apparatus in the kidney for help in controlling the diastolic and/or systolic blood pressure.

9. The synthetic polypeptide sequence of claim 1, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used as a tension activated pressure release valve in brain cells selected from the arachnoid granulations or the arachnoid cap cells for reducing intracranial hypertension.

10. The synthetic polypeptide sequence of claim 1, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used for neuromodulation in conjunction with external devices selected from ultrasound, photoacoustic or other physical methods for direct or indirect pressure-modulation.

11. The synthetic polypeptide sequence of claim 1, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used to reduce pain.

12. The synthetic polypeptide sequence of claim 11, wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, involves
a. Transfecting/transducing the pain sensory and processing neurons of peripheral/central nervous system including anterior cingulate cortex, and spinal cord; and
b. Activating transfected/transduced neurons at the targeted site in conjunction with external devices selected from ultrasound, photoacoustic or other physical methods for direct or indirect pressure-modulation.

13. The synthetic polypeptide sequence of claim 11, wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used to reduce pain generated by any kind of pressure by sensitizing inhibitory neurons, without the need of an external device for pressure-modulation.

14. The synthetic polypeptide sequence of claim 1, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used for improving viability and functioning of cells including neurons, endothelial, epithelial, muscular, cardiac cells.

15. The synthetic polypeptide sequence of claim 1, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used to suppress fibroproliferative activity.

16. The synthetic polypeptide sequence of claim 1, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used for therapeutic use without eliciting immune response.

17. The synthetic polypeptide sequence of claim 1, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants, are used for enhancing stimulation of cells, including neurons, cardiac and muscle cells for treatment and prevention of diseases selected from neurological diseases, epilepsy, stroke, cardiovascular diseases, muscular dystrophies in conjunction with external devices selected from ultrasound, photoacoustic or other physical methods for direct or indirect pressure-modulation.

18. The synthetic polypeptide sequence of claim 1, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants, are used as osmoregulators, activated by internal or external mechanical stimuli, in the central nervous system (CNS) mediated barriers selected from the blood-cerebrospinal fluid (CSF) barrier, the blood brain barrier (BBB), the blood-retinal barrier and the blood-spinal cord barrier.

19. The synthetic polypeptide sequence of claim 1, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used as a tension activated pressure release valve in cells in diseases characterized by elevated pressure or impaired permeability of tissues, selected from Glaucoma (Elevated Intraocular pressure), Stroke/Seizure (Intracranial pressure rise), Hypertensive encephalopathy (Elevated Hydrostatic force), Heart Failure (Interstitial tissue pressure), kidney disease (Interstitial Fluid accumulation), Erectile dysfunction (Blood flow abnormality), Lymphedema (Inadequate drainage of lymph fluid), Menopause (Stoppage of menstrual flow), and Hypertensive cardiomyopathy (Diastolic/systolic dysfunction).

20. The synthetic polypeptide sequence of claim 2, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains are delivered locally, intraocularly, intravenously, intrathecally, intramuscularly or subcapsularly.

21. The synthetic polypeptide sequence of claim 3, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains are delivered locally, intraocularly, intravenously, intrathecally, intramuscularly or subcapsularly.

22. The synthetic polypeptide sequence of claim 4, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains are delivered locally, intraocularly, intravenously, intrathecally, intramuscularly or subcapsularly.

23. The synthetic polypeptide sequence of claim 2, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used as a tension activated pressure release valve in cells for reduction of blood pressure.

24. The synthetic polypeptide sequence of claim 3, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used as a tension activated pressure release valve in cells for reduction of blood pressure.

25. The synthetic polypeptide sequence of claim 4, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used as a tension activated pressure release valve in cells for reduction of blood pressure.

26. The synthetic polypeptide sequence of claim 2, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used for neuromodulation in conjunction with external devices selected from ultrasound, photoacoustic or other physical methods for direct or indirect pressure-modulation.

27. The synthetic polypeptide sequence of claim 3, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used for neuromodulation in conjunction with external devices selected from ultrasound, photoacoustic or other physical methods for direct or indirect pressure-modulation.

28. The synthetic polypeptide sequence of claim 4, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used for neuromodulation in conjunction with external devices selected from ultrasound, photoacoustic or other physical methods for direct or indirect pressure-modulation.

29. The synthetic polypeptide sequence of claim 2, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used to reduce pain.

30. The synthetic polypeptide sequence of claim 3, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used to reduce pain.

31. The synthetic polypeptide sequence of claim 4, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used to reduce pain.

32. The synthetic polypeptide sequence of claim 2, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants, are used for enhancing stimulation of cells, including neurons, cardiac and muscle cells for treatment and prevention of diseases selected from neurological diseases, epilepsy, stroke, cardiovascular diseases, muscular dystrophies in conjunction with external devices selected from ultrasound, photoacoustic or other physical methods for direct or indirect pressure-modulation.

33. The synthetic polypeptide sequence of claim 3, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants, are used for enhancing stimulation of cells, including neurons, cardiac and muscle cells for treatment and prevention of diseases selected from neurological diseases, epilepsy, stroke, cardiovascular diseases, muscular dystrophies in conjunction with external devices selected from ultrasound, photoacoustic or other physical methods for direct or indirect pressure-modulation.

34. The synthetic polypeptide sequence of claim 4, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants, are used for enhancing stimulation of cells, including neurons, cardiac and muscle cells for treatment and prevention of diseases selected from neurological diseases, epilepsy, stroke, cardiovascular diseases, muscular dystrophies in conjunction with external devices selected from ultrasound, photoacoustic or other physical methods for direct or indirect pressure-modulation.

35. The synthetic polypeptide sequence of claim 2, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used as a tension activated pressure release valve in cells in diseases characterized by elevated pressure or impaired permeability of tissues, selected from Glaucoma (Elevated Intraocular pressure), Stroke/Seizure (Intracranial pressure rise), Hypertensive encephalopathy (Elevated Hydrostatic force), Heart Failure (Interstitial tissue pressure), kidney disease (Interstitial Fluid accumulation), Erectile dysfunction (Blood flow abnormality), Lymphedema (Inadequate drainage of lymph fluid), Menopause (Stoppage of menstrual flow), and Hypertensive cardiomyopathy (Diastolic/systolic dysfunction).

36. The synthetic polypeptide sequence of claim 3, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used as a tension activated pressure release valve in cells in diseases characterized by elevated pressure or impaired permeability of tissues, selected from Glaucoma (Elevated Intraocular pressure), Stroke/Seizure (Intracranial pressure rise), Hypertensive encephalopathy (Elevated Hydrostatic force), Heart Failure (Interstitial tissue pressure), kidney disease (Interstitial Fluid accumulation), Erectile dysfunction (Blood flow abnormality), Lymphedema (Inadequate drainage of lymph fluid), Menopause (Stoppage of menstrual flow), and Hypertensive cardiomyopathy (Diastolic/systolic dysfunction).

37. The synthetic polypeptide sequence of claim 4, for use in a method wherein heterologously expressed mechanosensitive channels, its variants, and the generated site-directed mutants thereof, optionally further including bacterial mechanosensitive channels, plant mechanosensitive channels (MSL2-10), TRPV1-TRPV5 channels, Piezo channels or their generated site-directed mutants derived from alternative strains, are used as a tension activated pressure release valve in cells in diseases characterized by elevated pressure or impaired permeability of tissues, selected from Glaucoma (Elevated Intraocular pressure), Stroke/Seizure (Intracranial pressure rise), Hypertensive encephalopathy (Elevated Hydrostatic force), Heart Failure (Interstitial tissue pressure), kidney disease (Interstitial Fluid accumulation), Erectile dysfunction (Blood flow abnormality), Lymphedema (Inadequate drainage of lymph fluid), Menopause (Stoppage of menstrual flow), and Hypertensive cardiomyopathy (Diastolic/systolic dysfunction).


SEQUENCE LISTING (9 sequences)

SEQ ID NO: 1 - protein, 157 aa, synthetic construct
  REGION 1..157: note = Synthetic Peptide
MLPQQVGFVCAVLALVCCASGMSIIKEFREFAMRGNVVDLAVGVIIGAAFGKIVSSLVADIIMPPLGLLIGGIDFKQFAVTLRDAQGDIPAVVMHYGVFIQNVFDFLIVAFAIFMAIKLINKLNRKKEEPAAAPAPTKEEVLLTEIRDLLKEQNNRS

SEQ ID NO: 2 - protein, 172 aa, synthetic construct
  REGION 1..172: note = Synthetic Peptide
MLPQQVGFVCAVLALVCCASGMLKGFKEFLARGNIVDLAVAVVIGTAFTALVTKFTDSIITPLINRIGVNAQSDVGILRIGIGGGQTIDLNVLLSAAINFFLIAFAVYFLVVLPYNTLRKKGEVEQPGDTQVVLLTEIRDLLAQTNGDSPGRHGGRGTPSPTDGPRASTESQ

SEQ ID NO: 3 - protein, 141 aa, synthetic construct
  REGION 1..141: note = Synthetic Peptide
MLPQQVGFVCAVLALVCCASGMLKEFKEFALKGNVLDLAIAVVMGAAFNKIISSLVENIIMPLIGKIFGSVDFAKEWSFWGIKYGLFIQSVIDFIIIAFALFIFVKIANTLMKKEEAEEEAVVEENVVLLTEIRDLLREKK

SEQ ID NO: 4 - protein, 157 aa, synthetic construct
  REGION 1..157: note = Synthetic Peptide
MLPQQVGFVCAVLALVCCASGMSLLKEFKAFASRGNVIDMAVGIIIGAAFGKIVSSFVADIIMPPIGIILGGVNFSDLSFVLLAAQGDAPAVVIAYGKFIQTVVDFTIIAFAIFMGLKAINSLKRKEEEAPKAPPAPTKDQELLSEIRDLLKAQQDK

SEQ ID NO: 5 - protein, 157 aa, synthetic construct
  REGION 1..157: note = Synthetic Peptide
MLPQQVGFVCAVLALVCCASGMSIIKEFREFAMRGNVVDLAVGVIIGAAFGKIVSSLVADIIMPPLGLLEGGIDFKQFAVTLRDAQGDIPAVVMHYGVFIQNVFDFLIVAFALFMAIKLINKLNRKKEEPAAAPAPTKEEVLLTEIRDLLKEQNNRS

SEQ ID NO: 6 - protein, 157 aa, synthetic construct
  REGION 1..157: note = Synthetic Peptide
MLPQQVGFVCAVLALVCCASGMSIIKEFREFAMRGNVVDLAVGVIIGAAFGKIVSSLVADIIMPPLGLLIGGIDFKQFAVTLRDAQGDIPAVVMHYGVFIQNVFDFLIVAFALFMAIKLINTLNRKKEEPAAAPAPTKEEVLLTEIRDLLKEQNNRS

SEQ ID NO: 7 - protein, 21 aa, synthetic construct
  REGION 1..21: note = Synthetic Peptide
MLPQQVGFVCAVLALVCCASG

SEQ ID NO: 8 - protein, 18 aa, synthetic construct
  REGION 1..18: note = Synthetic Peptide
MGRLLALVVGAALVSSAC

SEQ ID NO: 9 - protein, 18 aa, synthetic construct
  REGION 1..18: note = Synthetic Peptide
MAVPARTCGASRPGPART